(19) United States
(12) Patent Application Publication
(10) Pub. No.: US 2025/0110840 A1
(43) Pub. Date: Apr. 3, 2025

(54) SYSTEMS AND METHODS FOR SUPERVISING AND IMPROVING GENERATIVE AI CONTENT

(71) Applicant: Pieces Technologies, Inc., Irving, TX (US)

(72) Inventors: Rubendran AMARASINGHAM, Farmers Branch, TX (US); Yukun CHEN, Carrolton, TX (US); Josh GROB, Dallas, TX (US)

(21) Appl. No.: 18/905,459

(22) Filed: Oct. 3, 2024

Related U.S. Application Data
(60) Provisional application No. 63/587,528, filed on Oct. 3, 2023.
(60) Provisional application No. 63/667,386, filed on Jul. 3, 2024.

(51) Int. Cl. G06F 11/14; G06F 11/07

(52) U.S. Cl. CPC G06F 11/1476; G06F 11/0775

(57) ABSTRACT

A system, method, and a computer program product for detecting an error in artificial intelligence (AI) generated content. A neural network receives the AI generated content and at least one prompt and determines a concept in the AI generated content. Source content corresponding to the concept is retrieved. The neural network receives the source content, the concept, and a list of errors in a configuration file to determine an error in the AI generated content.

### CROSS-REFERENCE TO RELATED APPLICATIONS

This application claims priority to U.S. Provisional Application No. 63/587,528 filed on Oct. 3, 2023 and to U.S. Provisional Application No. 63/667,386 filed on Jul. 3, 2024, both of which are incorporated by reference in their entirety.

### TECHNICAL FIELD

This application relates generally to generative AI systems, and more specifically to detecting errors in the AI generated content of the generative AI system.

### BACKGROUND

Generative artificial intelligence (AI) produces new or novel content using neural networks. These systems have tremendous potential, but are also known to produce errors, and incorrect or false information, known as hallucinations. There is a need to detect and correct the generative AI output and retrain the generative AI systems to make the generative AI output more accurate.

### BRIEF DESCRIPTION OF THE DRAWINGS

FIG. 1 is a block diagram of an artificial intelligence error detection system for detecting errors in artificial intelligence generated content, according to some embodiments.

FIGS. 2-4 are diagrams of an interface of the artificial intelligence error detection system, according to some embodiments.

FIG. 5 is a simplified block diagram of an artificial intelligence hallucination detection module, according to some embodiments.

FIG. 6 illustrates an example computing device where embodiments may be implemented.

FIG. 7 is a simplified block diagram of a network system where embodiments may be implemented.

FIGS. 8A-B are diagrams of a method for identifying an error in artificial intelligence generated content, according to some embodiments.

FIG. 9 is a diagram of an artificial intelligence user interface, according to some embodiments.

FIG. 10 is a diagram of an artificial intelligence user interface, according to some embodiments.

FIGS. 11A and 11B are diagrams of an artificial intelligence user interface, according to some embodiments.

FIG. 12 is a diagram of a neural network for error detection in artificial intelligence generated content, according to some embodiments.

FIG. 13 is a diagram of a large language model for text generation and/or error detection, according to some embodiments.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

It will be readily understood that the components of disclosure, as generally described and illustrated in the figures herein, may be arranged and designed in a wide variety of different configurations. Thus, the following detailed description of the embodiments of a method, apparatus, and system, as represented in the attached figures, is not intended to limit the scope of the application as claimed, but is merely representative of selected embodiments of the application.

While AI systems may be adept at generating content, such as text summaries, from unstructured data or a combination of structured or unstructured data, AI systems may generate errors within the AI generated content. AI systems can make AI generated content up, also known as AI hallucination. Such errors may be fatal in high-risk settings. The embodiments are directed to an AI error detection system that detects errors in AI generated content and corrects errors within the AI generated content. The AI error detection system may be a stand-alone system that receives and analyzes the AI generated content on a computing device, in a client-server environment, or in a cloud system. Alternatively, the AI error detection system may be a plug-in into an AI system that generates content.

An AI error detection system may also facilitate finetuning of an AI system on specialized technical documents. AI systems are generally trained on a large corpus of texts, where the vast majority of text is unrelated to the specialize technical documents the AI system is being applied to. For example, the AI system may be tasked with generating text from medical reports, engineering reports, scientific papers in a specific field, etc. The AI error detection system may be specialized to a domain, e.g., medical, engineering, or science. In this way, the AI error detection system has an increased probability of finding errors related to the technical domain. Consequently, the AI system may be finetuned on the identified errors and/or resulting revisions to the generated text, which serve to retrain the AI system in a targeted manner and improve its performance in a technical domain.

In some embodiments, the output of an AI error detection system is a user interface structure that may be displayed on a display based on the display's physical characteristics (e.g., screen size, processing capability, and type of a display screen).

Further embodiments of the AI error detection system are discussed below.

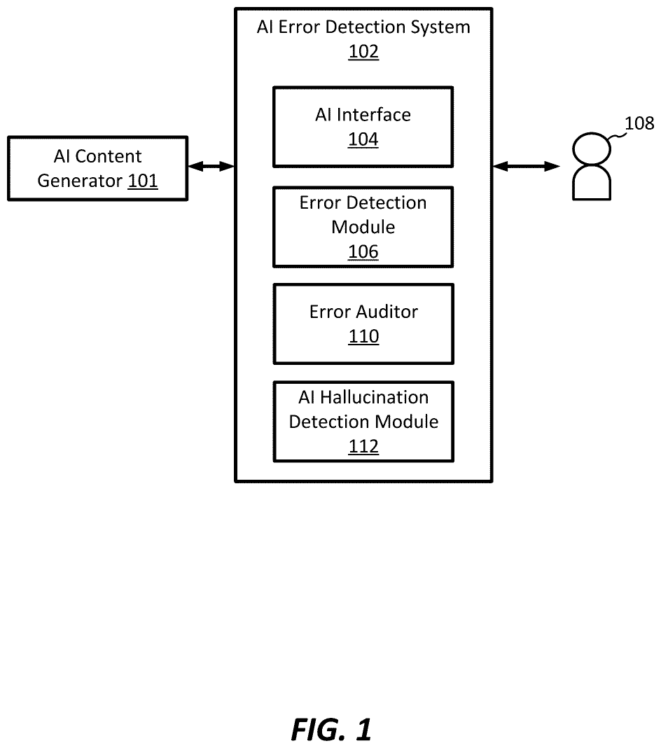

FIG. 1 is a diagram 100 of an artificial intelligence system, according to some embodiments. The artificial intelligence system may include an AI content generator 101 and AI error detection system 102. AI content generator 101 may include multiple neural networks that receive structured and unstructured data, also known as source content, and generate output, referred to as AI generated content. The AI generated content may be unstructured content such as text content, text summaries, figures, images or other forms of unstructured content. The AI generated content may also be structured content, such as data sheets, content formatted into rows and columns and the like, or a combination of structured and unstructured content. The AI generated content may be generated in real-time. As an example in the medical/clinical setting, AI generated content may be generated from source content that comprises any of the following in full or in part, including, AI generated clinical summaries, AI generated progress notes, AI generated history and physical notes, AI generated past medical history, AI generated radiology reports, AI generated operating notes, AI generated ambulatory progress notes, AI generated nursing notes, AI generated clinical flowsheet text, AI generated chat conversations between a patient and an AI system, AI generated patient questionnaires, and the like. As a further example in the medical/clinical setting, the AI generated content itself may be a clinical summary, patient summary, progress note, physician note, discharge barrier, history and physical notes, past medical history, radiology reports, operating notes, ambulatory progress notes, nursing notes, clinical flowsheet text, and the like.

As discussed above, AI generated content may include errors or AI hallucinations. These may be due AI content generator 101 being improperly trained, improperly finetuned, trained on training data that does not encompass the relevant use cases, data that is improperly or incompletely labelled, or any combination thereof. Errors may be when AI content generator 101 incorrectly represents information in the structured or unstructured data. AI hallucinations may be types of errors where AI content generator 101 created AI generated content that was not based on structured or unstructured data.

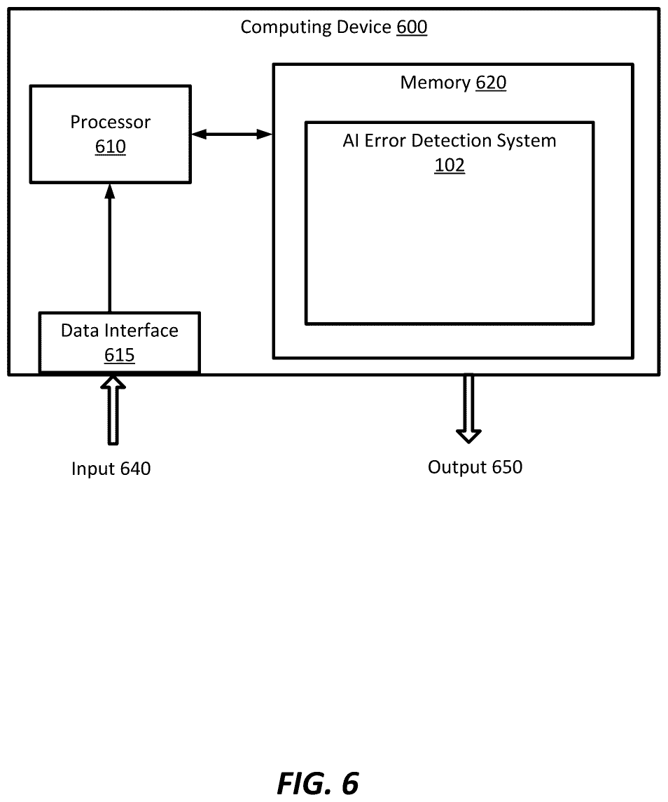

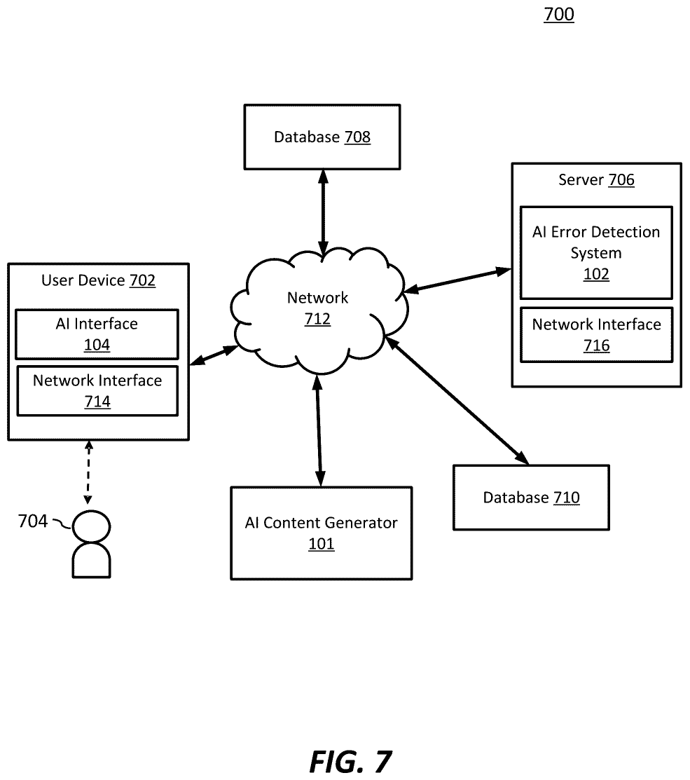

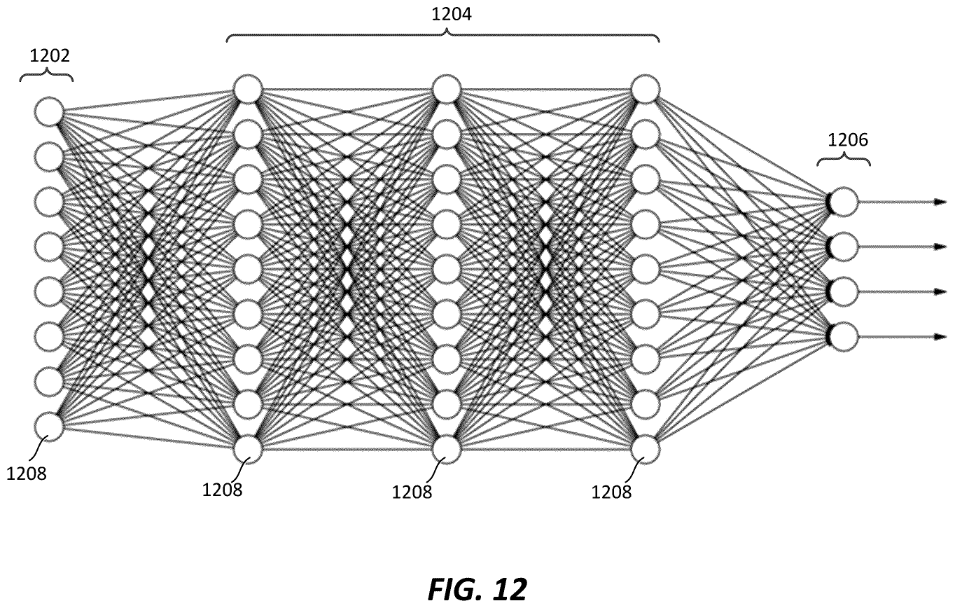

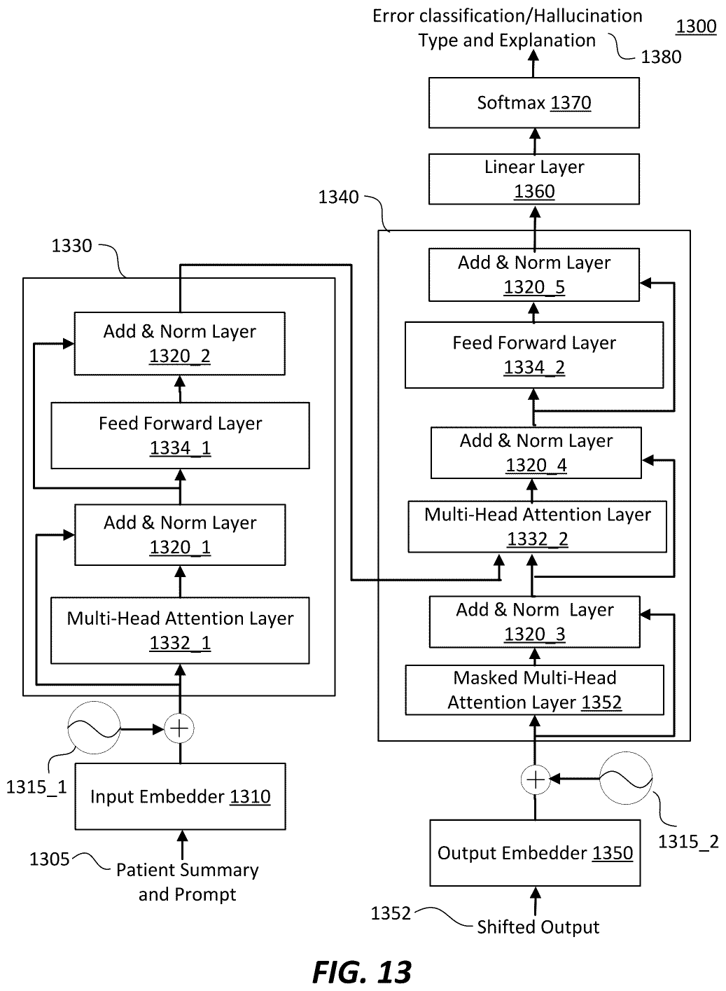

AI error detection system 102 may identify errors and hallucinations (collectively referred to as errors) in AI generated content. AI error detection system 102 may be software that is implemented using a combination of one or more computing devices, such as computing devices discussed in FIGS. 6 and 7. AI error detection system 102 may be implemented by one or more neural network models, such as those described in FIGS. 12-13. AI error detection system 102 may receive AI generated content in real-time or at predefined time intervals, modify and/or correct the AI generated content automatically and/or, in some instances, based on input from user 108. The modified AI generated content may correct errors in the AI generated content. Once modified, AI error detection system 102 may display the modified AI generated content and/or provide the modified AI generated content to AI content generator 101 to retrain or finetune the AI content generator 101 in real-time or at predefined time intervals.

AI error detection system 102 may include an AI interface 104 and an error detection module 106. AI interface 104 may display the AI generated content, e.g., AI generated a summary or summaries of patient records or another output for other use cases, and provide an interface for a review, editing, and modification of the AI generated content. Additionally, AI interface 104 may display source content, such as one or more documents, notes, text, images, etc., that were inputs based on which AI content generator 101 generated AI generated content. AI interface 104 may also receive input to filter and sort AI generated content, correct errors in AI generated content and the like.

In some instances, the user 108 may be an AI generated content reviewer, a clinician in a medical setting, a corporate officer in a commercial or corporate setting, and the like. User 108 may compare AI generated content to source content based on which AI content generator 101 generated AI generated content. User 108 may also be an automated script within error detection module 106 that may automatically compare AI generated content to source content to identify potential errors and flag those errors to, for example, a reviewer. User 108 may also activate AI error detection system 102 on demand or upon a notification or alert of potential errors in AI generated content.

Error detection module 106 may identify AI generated content that may include an error and classify the potential cause of the error. Error detection module 106 may also detect an error or a probability of an error within AI generated content according to the multi-part classifications, shown in more detail in Tables I-III, below. The classification may include different error types, such as the comprehension error types, the fluency error types, and the total AI content error type. The classification may also include a reason for the error, and a portion of source content that may be associated with the error. Error detection module 106 may be trained on the AI generated content, on the source content, and on the modified AI generated content to identify AI generated content that includes a potential error and classify the potential cause of the error. Alternatively, AI interface 104 may receive the classification for the error and the reason for the error from user 108, or provide a list of error classifications together with the error type. In some embodiments, error detection module 106 may classify the error based on the input received for modifying AI generated content via AI interface 104.

Table I, below, includes comprehension error types that may be generated and classified by the AI error detection system 102.

TABLE I
Comprehension Error Type (Quality of the input to Generative
Pre-trained Transformer (GPT) or Large Language Model)

| | | |
|---|---|---|
| Labeling | The concept identified was relevant to the | Pending Result: “4 . Follow - up x - |
| Error | comprehension task, but the wrong label | rays if patient's agrees.” - Should |
| | concept was assigned. | be Pending Test and Procedure |
| | | Transportation Needs: Will have a |
| | | hoyer lift at home. - Should be |
| | | Durable Medical Equipment |
| Domain | The concept identified was not relevant to | Pending Result: “Face sheet and |
| Error | the domain of this comprehension task, but | covid results attached.” |
| | could apply to a different domain. | |
| Concept | Relevancy error: The concept is valid but | Relevancy error: |
| Error | not related to any concept. | |
| | Gibberish error: The content was unclear | Gibberish error - Durable Medical |
| | or gibberish. | Device: “She is requesting a walker |
| | | and I negative except discharge at |
| | | the time of drinking change.” |
| | Completion error: The content is a broken | Completion error - Prior |
| | or incomplete sentence. | Authorization Need: “This source |
| | Context error: The content did not have | has not been approved by the.” |
| | enough context to assess. | |
| Parameter | Timing error: The concept identified was | Timing error (past) - Pending Test |
| Error | relevant to the comprehension task, but the | and Procedure: Atrial fibrillation s/p |
| | concept was either in the past or unknown | ablation |
| | timing in relation to present circumstances. | |
| | Attribution error. The concept identified | Attribution error (other) - sentence |
| | was relating to a person other than the | content is about another |
| | patient. | person/relative of the patient, not |
| | | the patient him/herself |
| | Negation error: The concept was negated. | Negation error - sentence content is |
| | | negating the concept it's labeled for |
| | Context error: The sentence content lacks a | Context error - sentence content |
| | verb or is a title. | lacks a verb or is a title - lacks |
| | | content for it to be a good input |
| PHI Error | Protected health information (PHI) was | |
| | included within the summary. | |

Table II, below, includes fluency error types that may be generated and classified by the AI error detection system 102. In this case, AI interface 104 may display the source content (input), AI generated content (output), and modified AI generated content (desired output).

TABLE II
Fluency Error Type (Quality of the GPT or LLM output)

| | | |
|---|---|---|
| Omission Error | The concept was not | INPUT: Insurance Related Issues: “ALL |
| | properly emphasized or | (PHILADELPHIA POSITIVE): She is |
| | prominently discussed or the | post a Haploidentical Allogeneic |
| | thought or sentence was not | transplant (mother) using FMT and |
| | properly finished or | ptCY in August 2015 at Hospital X, and |
| | completed or the original | due to insurance reasons, she is now |
| | concept was omitted. | following up with CAGT for post |
| | | transplant care.” |
| | | OUTPUT: Insurance Related Issues: |
| | | Patient is post a haploidentical |
| | | allogeneic transplant and is now |
| | | following up with CAGT for post |
| | | transplant care. |
| | | DESIRED OUTPUT: Post-Discharge |
| | | Placement: Due to insurance reasons, |
| | | patient is now following up with CAGT |
| | | for post haploidentical allogeneic |
| | | transplant care. |
| | | INPUT: Post-Discharge Placement: “PT/ |
| | | OT are recommending possible IRF, |
| | | but patient has only medicaid.” |
| | | OUTPUT: Post-Discharge Placement: |
| | | Patient has only medicaid. |
| | | DESIRED OUTPUT: Post-Discharge |
| | | Placement: PT/OT recommends IRF but |
| | | patient has only medicaid. |
| Mistranslation | The output did not properly | INPUT: Behavioral Challenge: “Seizure |
| Error | describe the original concept | precautions, sitter at bedside.” |
| | or the intent of the concept. | OUTPUT: Behavioral Challenge: Patient |
| | | requires seizure precautions due to |
| | | having a sitter at bedside. |
| | | DESIRED OUTPUT: Behavioral |
| | | Challenge: Seizure precautions: sitter at |
| | | bedside. |
| | | INPUT: Total Parenteral Nutrition |
| | | (TPN): “malnourished; severe; attempt |
| | | at PEG tomorrow; if fails, then home on |
| | | TPN - right chest wall cellulitis; on |
| | | vancomycin.” |
| | | OUTPUT: Parenteral Nutrition (TPN): |
| | | Patient will require PEG if fails. |
| | | DESIRED OUTPUT: Total Parenteral |
| | | Nutrition (TPN): Try PEG tomorrow; if |
| | | fails, will discharge home on TPN. |
| Elaboration Error | Too many words or details | INPUT: DME Oxygen: “BCBS Case |
| (also called | included from the original | Manager stated she will email this CM a |
| “Hallucination | concept, or new details added | list of in - network vendors if home |
| Error”) | into the summary, that were | oxygen needs to be arranged.” |
| | beyond the original concept. | OUTPUT: DME Oxygen: Will email list |
| | | of in-network vendors if home oxygen |
| | | needs to be arranged. |
| | | DESIRED OUTPUT: DME Oxygen: |
| | | Patient may need home oxygen. |
| Grammar Error | The output was not | INPUT: Durable Medical Device: “SW |
| | grammatically correct, | placed referral via parachute to adapt |
| | including spelling, | health for dme to be delivered to patients |
| | capitalization, tense, and | house.” |
| | syntax. | OUTPUT: Durable Medical Device: SW |
| | | placed referral via parachute to adapt |
| | | health for dme to be delivered to patients |
| | | house. |
| | | DESIRED OUTPUT: Durable Medical |
| | | Device: SW placed referral to Adapt |
| | | Health for DME to be delivered to |
| | | patient's house. |
| Medical | Intensity error: A new | INPUT: Pending Consult: “Neuro optho |
| Misinterpretation | medical concept was | consult/follow up pending.” |
| Error | swapped with the original | OUTPUT: “Pending Consult: Awaiting |
| | medical concept; the wrong | neurosurgery recommendations.” |
| | medical nomenclature was | DESIRED OUTPUT: Neuro optho was |
| | used; or the adjectives | consulted; awaiting recommendations. |
| | describing the concept were | INPUT: Voiding Status: “resume current |
| | either too low or high in | dose of IV diuretics for one additional |
| | intensity. | day and wean O2 as tolerated - ok to d/ |
| | | c foley but will need q6h pvr to make |
| | | sure no retention and strict I/O.” |
| | | OUTPUT: Voiding Status: Plan to |
| | | discontinue foley but will need to |
| | | continue daily piperacillin/vancocin and |
| | | IV diuretics for one additional day. |
| | | DESIRED OUTPUT: Voiding Status: |
| | | Plan to discontinue foley; will need PVR |
| | | every 6 hours to make sure no retention |
| | | and strict I/O. |
| Toxicity error | Misogynistic, violent, racist, | |
| | sexist language or | |
| | conclusions mentioned. | |

Table III, below, includes summarization error types that may be generated and classified by the AI error detection system 102.

TABLE III
Total Summarization Error Type

| | |
|---|---|
| Smoothing error | The individual tasks were not smoothly integrated |
| | within the summary. |
| Priority Error | The concept identified was relevant to the |
| | comprehension task, but was not important enough to |
| | be prioritized into the input or the user. |
| Omission error | The concepts were not properly emphasized or |
| | prominently discussed or the thought or sentence was |
| | not properly finished or completed or the original |
| | concepts were omitted. |
| Elaboration error | Too many words or new concepts added that were |
| | beyond the original concepts. |
| Mistranslation | The output did not properly describe the original |
| Error | concepts or the intent of those concepts. |
| Medical | A new medical concept was swapped with the original |
| Misinterpretation | medical concept. |
| Error | The wrong medical nomenclature was used. |
| | Intensity error: the adjectives describing the concept |
| | were either too low or high in intensity. |
| Grammar error | The summary was not grammatically correct, including |
| | spelling, capitalization, tense, and syntax. |

One of ordinary skill in the art would appreciate that the errors and error types listed in Tables I-III above for the medical AI generated content may also apply to errors in other settings, including financial record summaries, construction summaries, fraud detection or identification summaries, network device connectivity summaries and the like. Error detection may also be applied to information in other formats, such as figures, charts, graphs, and spreadsheets that may be generated using AI models. Errors and error types may be stored in a configuration file that is accessible to error detection module 106. Further, neural networks within error detection module 106 may be trained to identify and classify errors included in the configuration file.

In some embodiments, error detection module 106 may operate in an automated, or partially automated mode. Error detection module 106 may comprise one or more neural networks, such as those described in FIGS. 12-13. For example, error detection module 106 may employ an adversarial AI network and/or other types of neural networks, such as large language models (LLMs) to detect an error in AI generated content. An adversarial AI networks may include multiple different network architectures, such as text-based generative adversarial networks, and learning paradigms, such as contrastive learning. Adversarial AI network may use one network to generate AI generated content and another network to determine if an error was made by the first network. Said another way, adversarial AI network may pit two different networks against each other to improve the accuracy and robustness of the AI generated content.

Error detection module 106 may receive AI generated content, such as AI generated summaries and one or more predefined prompts. The predefined prompts may be stored within the same or different configuration file as the errors and error types. Using the AI generated content and prompts, error detection module 106 may cause the large language model or other neural network(s) to parse AI generated content into one or more constituent concepts, treatments, etc., specified in the prompts. For example, AI generated content that is a patient summary may include vital signs, laboratory results for a patient, and a prescribed treatment plan, which the error detection module 106 may parse into three separate identified concepts/treatments. In some instances, concepts may be identified at varying levels of detail, e.g., extensive laboratory results may be further split into a variety of separate concepts such as laboratory results for a particular organ function, blood results, urinalysis, and the like. In another instance, concepts may be prioritized, such that concepts that have a higher priority (such as heart attack, sepsis shock, etc.) may be identified and concepts that have lower priority (such as headache) may be ignored. The concept priority may also be stored in the same or different configuration file as errors and error types and prompts.

In some embodiments, using concepts parsed from the AI generated content, error detection module 106 may determine whether the AI generated content is valid, has an error, or is a hallucination. For example, error detection module 106 may query a database, e.g., 708 and 710 described in FIG. 7, containing source content. Error detection module 106 may identify source content, such as notes, patient electronic medical record (EMR), patient electronic health record (EHR), and any other associated medical records, including physician's notes, that are related to a parsed concept. After identifying any relevant source content, error detection module 106 may compare the concept(s) and/or treatments from the AI generated content with the information available in the identified source content. If the information in the AI generated content does not match the identified source content, error detection module 106 may flag the AI generated content as an error. Error detection module 106 may then pass the AI generated content and the identified source content through the large language model or neural network to determine a type of an error. Further, error detection module 106 may add the AI generated content to a queue of other AI generated content that has error for further review. The queue may be viewable by a user, such a medical professional/clinician, through an AI interface 104.

In some embodiments, error detection module 106 may generate a confidence score indicative of the certainty that the comparison is correct. For example, if an identified source note was identified as relevant for laboratory results present in AI generated content but the source content contained no such results, then the comparison is likely to result in identifying an error in the AI generated content. However, that error probably reflects a failure to find the relevant source content, so error detection module may indicate a low confidence score, e.g., <50%.

In some embodiments, AI error detection system 102 may assign user 108, e.g., a reviewer, to a specific subset of the AI generated content. For example, AI error detection system 102 may include user identifiers that correspond to a list of reviewers, and may allocate to each user identifier a subset of the AI generated content and corresponding source content. Once the reviewer accesses the AI error detection system 102, AI interface 104 may display the subset of AI generated content assigned to the reviewer.

In some embodiments, AI interface 104 provides multiple filtering functions. The filtering functions may receive input that selects AI generated content for review based on key words in the content, error profile, note type, and reviewer (e.g., user 108) background. For example, when AI error detection system 102 receives a user identifier of a user 108 having a background in radiology, the AI error detection system 102 may identify AI generated content that is specific to radiology and provide a listing of the AI generated content on AI interface 104 that includes content associated with radiology. In some instances, AI error detection system 102 may create a review block. The review block is set of cases or projects to review by one or more assigned reviewers. The filtering functions may be configured using AI interface 104 or via an automated script based on a profile or user identifier of the reviewer.

In some embodiments, error detection module 106 may include an AI model that compares the AI generated content to the source content and identifies potentially incorrect text in the AI generated content. AI interface 104 may display the AI generated content and/or source content and may highlight the areas that AI error detection system 102 may have identified as incorrect. The highlighted text may be based on the AI's understanding of the AI generated content. In some embodiments, error detection module 106 may also provide reasoning, explains the error type and why the AI generated clinical text may be inaccurate in plain English language (or another language) for the reviewer to evaluate.

In some embodiments, AI interface 104 may receive input from the user 108 that may modify the AI generated text. The modification may be based on the source content that AI interface 104 displays with the AI generated content. Once AI interface 104 receives the AI generated text, error detection module 104 may generate tags, e.g., add metadata to the source content that may be used to train AI content generator 101 to generate AI generated content.

AI error detection system 102 may also save or store the modified AI generated content into a database or another memory storage. In some instances, AI error detection system 102 may also assign a flag to the modified AI generated content that indicates that the modified AI generated content should not be overwritten, e.g., by the AI content generator 101 generating new AI generated content for the same patient. The flag may be set for a predefined time period, such as a day, a few hours, and the like.

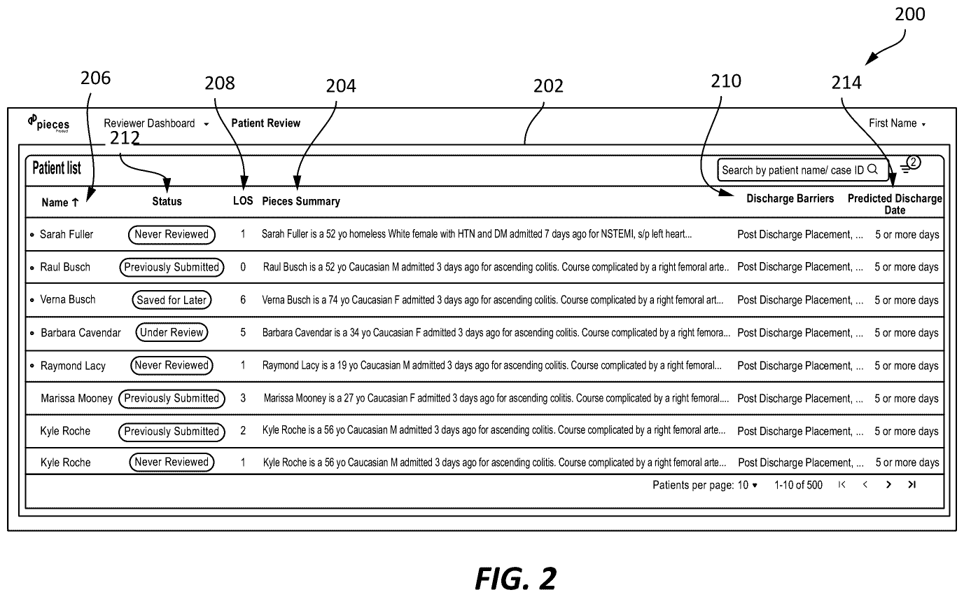

FIGS. 2-4 depict user interface capable of displaying AI generated content and/or corrected AI generated content generated by the AI error detection system 102. User interface may be configured to size and layout the presentation of the material based on the screen size, device, or other settings of the device being operated by a user. For example, one or more rows or columns described with respect to FIGS. 2-4 may be removed from the user interface when a user device is detected to be a mobile phone, tablet, or other device with a smaller screen. Alternatively, if the display screen is larger, e.g., laptops, large-panel monitors, additions rows and columns of information may be included on the user interface for display. Settings associated with a user's credentials (username, password, name, ID number, etc.) may cause the user interface to prioritize certain rows and columns of information over others for display. For example, the “Note View” column may be less relevant and thus removed from the user interface. In another example, a user's credentials may identify the user type, e.g., nurse or physician, and the user interface may display rows and columns of data relevant to the user type. For example, recent treatment of a patient may be more relevant for a nurse than a surgeon who may be evaluating the extended medical history of a patient.

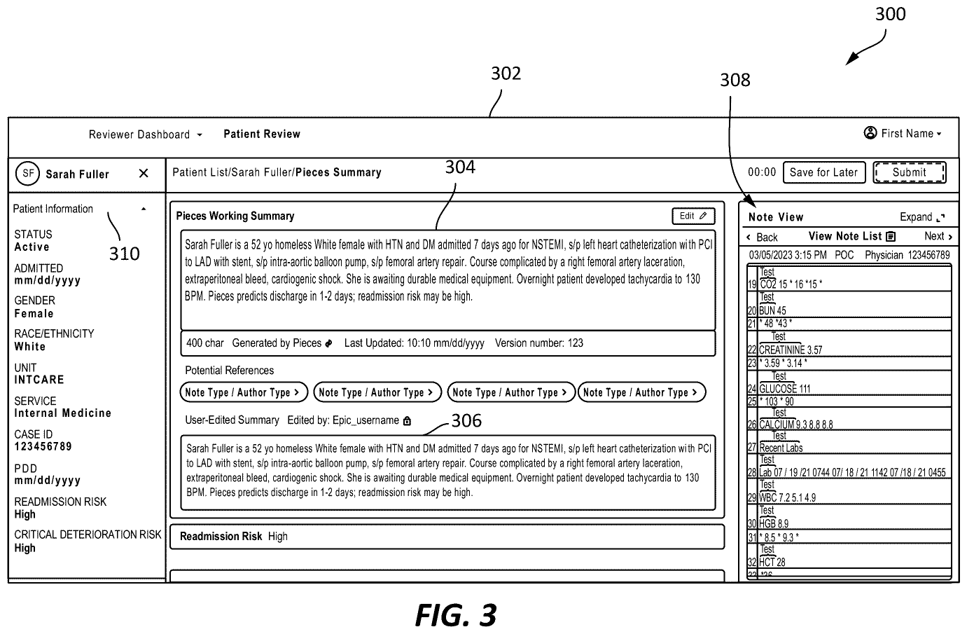

Furthermore, in some embodiments, a user is able to alter the size of various sub-windows containing the rows and columns of information directly through a user input device such a keyboard, mouse, trackpad, etc. For example, the “Note View” sub-window shown in FIG. 3 may be resized by receiving input that moves a cursor to one of the edges of the sub-window, receiving input through a mouse button, and receiving input for moving the sub-window. Other sub-windows may automatically resize to accommodate the input for resizing of another sub-window.

FIG. 2 is a diagram 200 of an artificial intelligence user interface, according to some embodiments. FIG. 2 may include an AI user interface 202 which may be AI interface 104. AI user interface 202 may receive and display AI generated content associated with multiple patients. AI user interface 202 may format the AI generated content in a list or another format. For example, AI user interface 202 may display a summary view of the patient data from multiple patients. The patient data may include a portion of AI generated content, such as a portion of the text summary in column 204 and patient names that corresponds to the AI generated content included in the name column 206. Additionally, AI user interface 202 may include a status column 212 that indicates the review status of the AI generated content. The review status may indicate whether the AI generated content was previously reviewed by user 108 (e.g., the reviewer) or error detection module 106, is under review, is saved for latter, or previously submitted by a reviewer.

AI user interface 202 may also include a discharge barrier column 210. The discharge barrier column displays the reasons or a portion of the reasons, if any, that prevent discharge of the patient as determined by the AI content generator 101. Additionally, AI user interface 202 may include a length-of-stay (LOS) column 208 that indicates how long a patient has been at a medical facility since their most recent admission, and projected discharge date column 214 that indicates the patient's projected discharge date.

The data displayed in the AI user interface 202 may be sorted or searched based on the information displayed in the various columns 204-214. For example, AI user interface 202 may receive input for sorting data based on the discharge barrier column 210 or projected discharge date column 214 to identify AI generated content that may be more time sensitive for review than other AI generated content.

The AI generated content may be uploaded to AI user interface 202 from a database or another memory storage. The AI generated content may be uploaded when AI user interface 202 is accessed or AI error detection system 102 is activated. Additionally, AI user interface 202 may be updated in real-time or at predefined time intervals when new AI generated content is saved to the database by, e.g., AI content generator 101.

Notably, the columns 204-214 displayed by AI user interface 202 are exemplary, as the columns of AI user interface 202 may be configured to reflect the project name, status, project duration, or summary of current status for use cases in other industries. For example, a construction company may have several ongoing construction projects at varying stages of completion and faced with varying challenges (e.g., labor shortages, city ordinance restrictions, or extended/delayed contractual negotiations).

FIG. 3 is a diagram 300 of an artificial intelligence user interface, according to some embodiments. AI user interface 302 may receive input from a user, such as a reviewer, and display AI generated content. AI user interface 302 may be accessed from the AI user interface 202 by selecting data displayed in AI user interface 202, such as a portion of the text summary in column 204. AI user interface 302 shown in FIG. 3 may display data associated with a patient, including AI generated content. An example of the AI generated content may be a summary of the patient's conditions. AI generated content may be displayed in a first text box or a window 304. In some instances, AI user interface 302 may highlight a portion of AI generated content that error detection module 106 identified as having an error. Additionally, AI user interface 302 may include a window or a text box 306 where AI generated content 306 may be modified, such as a modified AI generated summary of the patient conditions. The second window or text box 306 may receive input via a stylus, keyboard, or another input device that may modify the AI generated content. Additionally, the second window or text box 306 may receive the modified AI generated content that was automatically modified using error detection module 106. After modifications, AI user interface 302 may submit the modified AI generated content to AI content generator 101 discussed above, for subsequent training or finetuning.

In some embodiments, AI user interface 302 may include a note view 308. Note view 308 may display source content in a third text box or a window. The notes may be source content received over a network, e.g., original notes as entered by the treating physician or nurse, images of the notes (not shown), or a subset of data in the notes that was tagged as relevant to determining AI generated content displayed in the text box 304.

AI user interface 202 may include an information panel 310 in another text box or window. Information panel 310 may display patient information, as depicted in FIG. 3, or other types of information for various use cases. It should be appreciated that any combination of any subset of text boxes or windows described herein may be implemented.

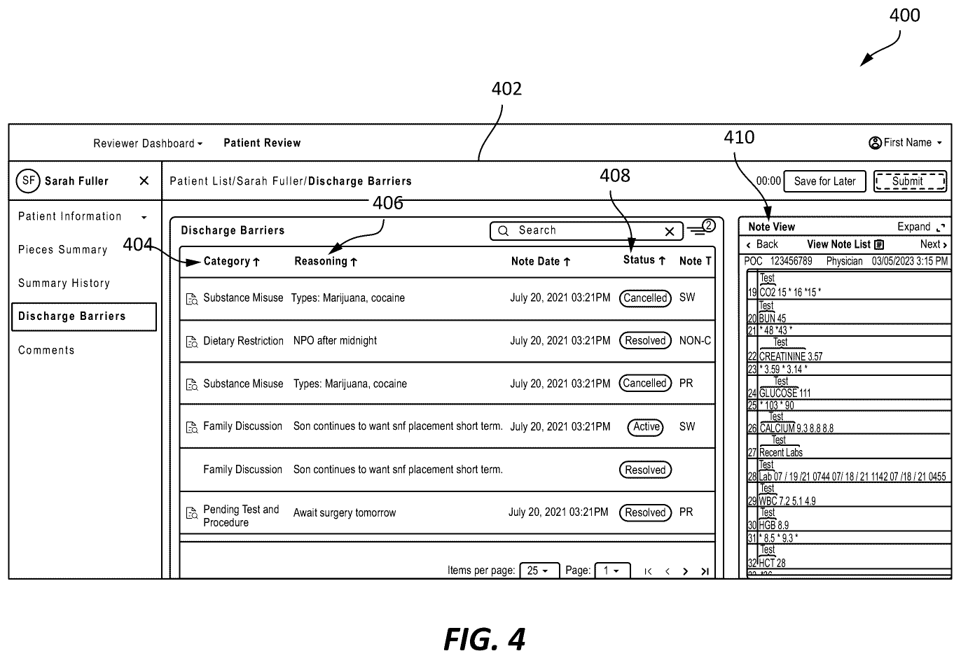

FIG. 4 is a diagram 400 of an artificial intelligence interface, according to some embodiments. AI user interface 402 shown in FIG. 4 may include data associated with the barriers to discharge. AI user interface 402 may be accessed from the AI user interface 202 or AI user interface 302 by selecting a link activating AI user interface 402. AI user interface 402 may display patient data, including category column 404 displaying one or more categories with a particular discharge barrier, e.g., “Substance Misuse,” “Dietary Restriction,” and the like. The barriers to discharge may be predicted by the AI content generator 101 discussed above, and uploaded to AI user interface 402 from a database when AI user interface 402 is accessed or AI error detection system is activated.

AI user interface 402 may also include a reasoning column 406. Reasoning column may display reasons for each barrier to discharge specified in column 404. Example barriers to discharge may include “NPO after midnight,” “Types: Marijuana, cocaine,” and the like. The information in the reasons column 402 may also be generated or identified by the AI content generator 101 and uploaded to AI user interface 402 from a database. Additionally, AI user interface 402 may include a current status 408, such as the current status of the barriers to discharge, such as “Resolved,” “Cancelled,” “Active.” In some instances, the reviewing medical professional may change the status of the barrier to discharge and further provide reasons for the change which may be used to finetune the AI model(s) in the AI content generator 101.

In some instances, AI user interface 402 may include a note view 410. Note view 410 may include source content based on which AI content generator 101 determined the barriers to discharge. Moreover, the source content may be tagged to display a subset of data in the source content that was determinative to the barrier to discharge or the reasons to the barrier to discharge. The tags may be generated or set by the AI content generator 101 or the AI error detection system 102.

The data in the AI user interface 402 may also be sorted or searched based on the information displayed in the various columns 404-408. When AI user interface 402 receives input to sort one of the columns 404-408, AI user interface 402 may sort the rows in all of the columns 404-408, with the sort based on the selected column.

In some embodiments, the columns of AI user interface 402 may reflect the project name, status, completion barrier, etc. for use cases in other industries. For example, a construction company may have several ongoing construction projects at varying stages of completion and faced with varying challenges (e.g., labor shortages, city ordinance restrictions, or extended/delayed contractual negotiations).

Going back to FIG. 1, in some embodiments, AI error detection system 102 may include an error auditor 110. Error auditor 110 may randomly select a list of AI generated content associated with multiple patients that has been reviewed and modified by user 108 (e.g., the reviewer). Error auditor 110 may present the list of the reviewed AI generated content on AI interface 104. Another user 108 may parse the list and assign a score to each reviewed AI generated content, where the score corresponds to a severity of the error in the modified content, if any. The score may be based on a predetermined score scale, color scale, and the like. Error auditor 110 may then perform analytics on the scores and generate a report which may represent an accuracy of the AI error detection system 102. The report may identify the errors in the AI generated content that was generated by AI content generator 101, the errors in the reviewed AI generated content reviewed by a particular reviewer or multiple reviewers, the errors in AI generated content by error type, classification type, or content type, e.g., by summary, by barriers to discharge, and the like.

In some embodiments, error auditor 110 may include software, e.g., an automated script, that tracks and evaluates the reviewer's (user 108) progress, speed of reviewing the AI generated content, reviewer agreement and quality of the reviewed AI content. In this way, error auditor 110 may also generate analytics that relate the number and types of errors as a function of user 108 and the speed of reviewing the AI generated content, and the like.

In some embodiments, AI error detection system 102 includes an AI hallucination detection module 112. AI hallucination module 112 may identify hallucinations in AI generated content. Identifying hallucinations may be particularly important in high-risk AI generated content that could have severe downstream effects. Additionally, AI hallucination module 112 may identify hallucinations in source content that AI content generator 101 may misinterpret, incorrectly summarize, and the like. AI hallucination detection module 112 may be software, incorporate a set of rules, scripts, configuration files, and the like.

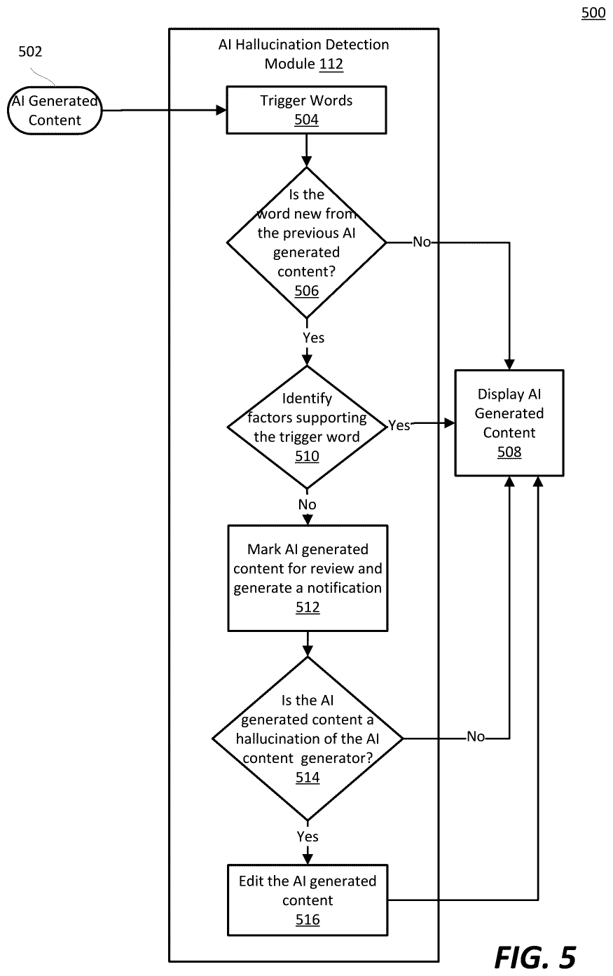

FIG. 5 is a block diagram 500 of a hallucination module, according to some embodiments. AI hallucination detection module 112 may include or access a configuration file in a memory storage that includes trigger words 504. Trigger words 504 may be indicative of a severity of a patient's condition. For example, trigger words may look for words such as “sepsis,” “stroke,” “hemorrhage,” etc. The list of trigger words may be available as a pre-defined list in the configuration file. In some instances, the list of trigger words may be facility or practice specific. In some instances, the list of trigger words may be provided by medical professionals and/or clinicians. In some instances, the list of trigger words may be updated in real-time by modifying the configuration file. Alternatively, AI hallucination detection module 112 may upload the trigger words from a database or elsewhere from another memory storage.

AI hallucination detection module 112 may receive AI generated content 502 and use natural language processing or rules to compare one or more words in AI generated content 502 to the trigger words 504. If AI hallucination detection module 112 determines that there is a match between the words in AI generated content 502 and trigger words 504, AI hallucination detection module 112 may access a previous instance of AI generated content 502 and determines if the previous instance of AI generated content 502 includes the same trigger word at step 506. If so, AI hallucination detection module 112 causes AI interface 104 to display the AI generated content 502 at step 508. If not, AI hallucination detection module 112 identifies supporting factors for the word or words that matched the trigger words 504. The supporting factors may be included in a configuration file and may be associated with trigger words 504. Alternatively, user 108 may review AI generated content 502 and provide AI hallucination detection module 112 with supporting factors at step 510. If supporting factors are identified then the AI generated content 502 may be displayed in step 508 as discussed above. If supporting factors are missing, AI hallucination module 112 marks the AI generated content 502 for review at step 512. Additionally, at step 512, AI hallucination module 112 may generate an alert, e.g., send a text message, and email or open an AI interface 104 discussed in FIG. 1. Upon reviewing AI generated content 502, if the medical professional determines the AI content is not an error or a hallucination based on the known information, then the AI content may be displayed in step 508. If the medical professional determines the AI content includes an error or a hallucination at step 514, then AI interface 104 may receive input that edits the AI generated content at step 516. After step 516, the edited AI generated content may be transmitted back to AI content generator 101 (not shown), to finetuning the AI content generator. Additionally, the edited AI generated content may be displayed in step 508. In this way, hallucinations by the AI models within AI content generator 101 may be corrected.

In some instances, the modified AI generated content 502 may be saved to a database with a flag that indicates that the modified AI generated content 502 should not be overwritten for a predefined time period by AI content generator 101.

In some embodiments, the identification of one or more trigger words 504 in AI generated content may trigger the automated review by the error detection module 106, as described herein.

In some embodiments, AI generated content may become stale or out-of-date due to the availability of new source content. When new source content becomes available, an updated AI generate content may be generated. The updated AI generated content may be reviewed as described herein, e.g., by the error detection module 106 and/or AI hallucination module 112. If previously error detection module 106 detected an error in the AI generated content and placed it in a queue for review and the updated AI generated content does not have a detected error, then the original AI generated content may be removed from the queue for review and re-run using error detection module 106. Alternatively, if the updated AI generated content is reviewed and an error is found, then it may replace the AI generated content in the queue and/or be added to the queue.

In a medical setting, a stream of medical documents and reports may be generated during the course of operation of a medical facility, such as a hospital. In some instances, this could mean tens or hundred of thousands of pages of medical information generated each day. The documents and reports may be stored in a database as described in FIG. 7, and then transferred, via network, to the AI content generator 101. In this way, AI content generator 101 may produce a large body of AI generated-content which is evaluated by the AI error detection system 102. Consequently, the AI error detection system 102, and one or more of its components such as AI hallucination detection module 112, are reviewing all of the AI-generated content at a rate beyond any human reviewer. Similar considerations would apply to the documents produced in other settings and industries.

FIG. 6 is a simplified diagram illustrating a computing device implementing the artificial intelligence user interface described in FIGS. 1-5 and other embodiments described herein. As shown in FIG. 6, computing device 600 includes a processor 610 coupled to memory 620. Operation of computing device 600 is controlled by processor 610. And although computing device 600 is shown with only one processor 610, it is understood that processor 610 may be representative of one or more central processing units, multi-core processors, microprocessors, microcontrollers, digital signal processors, field programmable gate arrays (FPGAs), application specific integrated circuits (ASICs), graphics processing units (GPUs) and/or the like in computing device 600. Computing device 600 may be implemented as a stand-alone subsystem, as a board added to a computing device, and/or as a virtual machine.

Memory 620 may be used to store software executed by computing device 600 and/or one or more data structures used during operation of computing device 600. Memory 620 may include one or more types of machine-readable media. Some common forms of machine-readable media may include floppy disk, flexible disk, hard disk, magnetic tape, any other magnetic medium, CD-ROM, any other optical medium, punch cards, paper tape, any other physical medium with patterns of holes, RAM, ROM, PROM, EPROM, FLASH-EPROM, any other memory chip or cartridge, and/or any other medium from which a processor or computer is adapted to read. Further, the above embodiments may be implemented in hardware, in a computer program executed by a processor, in firmware, or in a combination of the above. A computer program may be embodied on a computer readable medium, such as a storage medium. For example, a computer program may reside in random access memory (“RAM”), flash memory, read-only memory (“ROM”), erasable programmable read-only memory (“EPROM”), electrically erasable programmable read-only memory (“EEPROM”), registers, hard disk, a removable disk, a compact disk read-only memory (“CD-ROM”), or any other form of storage medium known in the art.

Processor 610 and/or memory 620 may be arranged in any suitable physical arrangement. In some embodiments, processor 610 and/or memory 620 may be implemented on a same board, in a same package (e.g., system-in-package), on a same chip (e.g., system-on-chip), and/or the like. In some embodiments, processor 610 and/or memory 620 may include distributed, virtualized, and/or containerized computing resources. Consistent with such embodiments, processor 610 and/or memory 620 may be located in one or more data centers and/or cloud computing facilities.

In some examples, memory 620 may include non-transitory, tangible, machine readable media that includes executable code that when run by one or more processors (e.g., processor 610) may cause the one or more processors to perform the methods described in further detail herein. For example, as shown, memory 620 includes instructions for AI error detection system 102 that may receive input 640 such as an AI generated content via the data interface 615 and generate an output 650 which may be edited, marked, modified, or otherwise manipulated AI generated content, audit reports, etc.

The data interface 615 may comprise a communication interface, a user interface (such as a voice input interface, a graphical user interface, and/or the like). For example, the computing device 600 may receive the input 640 (such as an AI-generated content) from a networked database via a communication interface. Or the computing device 600 may receive the input 640, such as corrections to an AI-generated content, from a user via the user interface.

FIG. 7 is a simplified block diagram of a networked system 700 suitable for implementing the framework described in FIGS. 1-5 and other embodiments described herein. In one embodiment, system 700 includes the user device 702 which may be operated by user 704, server 706, and multiple databases, such as databases 708 and 710, and other forms of devices, servers, and/or software components that operate to perform various methodologies in accordance with the described embodiments. Exemplary devices and servers may include device, stand-alone, and enterprise-class servers which may be similar to the computing device 600 described in FIG. 6, operating an OS such as a MICROSOFT® OS, a UNIX® OS, a LINUX® OS, or other suitable device and/or server-based OS. It can be appreciated that the devices and/or servers illustrated in FIG. 7 may be deployed in other ways and that the operations performed, and/or the services provided by such devices and/or servers may be combined or separated for a given embodiment and may be performed by a greater number or fewer number of devices and/or servers. One or more devices and/or servers may be operated and/or maintained by the same or different entities.

The user device 702, server 706, and databases 708 and 710 may communicate with each other over a network 712. Network 712 may be implemented as a single network or a combination of multiple networks. For example, in various embodiments, network 712 may include the Internet or one or more intranets, landline networks, wireless networks, and/or other appropriate types of networks. Thus, network 712 may correspond to small scale communication networks, such as a private or local area network, or a larger scale network, such as a wide area network or the Internet, accessible by the various components of system 700.

User device 702 may receive input from a user 704 (e.g., a driver, a system admin, etc.) to access the various features available for user device 702, which may include processes and/or applications associated with the server 706. User 704 may be user 108 discussed in FIG. 1 or other users discussed above.

User device 702 may be implemented as a communication device that may utilize appropriate hardware and software configured for wired and/or wireless communication with server 706. For example, user device 702 may be implemented as a personal computer (PC), a smart phone, laptop/tablet computer, wristwatch with appropriate computer hardware resources, eyeglasses with appropriate computer hardware (e.g., GOOGLE GLASS®), other type of wearable computing device, implantable communication devices, and/or other types of computing devices capable of transmitting and/or receiving data. Although only one communication device is shown, a plurality of communication devices may function similarly.

User device 702 may contain AI interface 104 discussed in FIG. 1 in a client-server implementation of AI error detection system 102. For example, AI interface 104 may communicate over network 712 with other components of AI error detection system 102 that, for example, execute elsewhere on server 706. To communicate, user device 702 may include at least one network interface component 714 adapted to communicate with server 706, and databases 708, 710. In various embodiments, network interface component 714 may include a DSL (e.g., Digital Subscriber Line) modem, a PSTN (Public Switched Telephone Network) modem, an Ethernet device, a broadband device, a satellite device and/or various other types of wired and/or wireless network communication devices including microwave, radio frequency, infrared, Bluetooth, and near field communication devices.

Database 708 may store AI generated content, including AI-generated summaries, generated by AI content generator 101. For example, AI content generator 101 may transmit AI generated content over network 712 to database 708 in real-time (e.g., as soon as AI generated content is generated), or at predefined time intervals. Database 708 may also store source content, or a subset of source content, e.g., content AI content generator 101 used to generate AI generated content. In some instances, source content that has contributed substantially (e.g., as determined by AI content generator 101, a reviewer, or the like) may be labeled or include metadata that identifies the source content to contributing content. Database 708 may be implemented by one or more relational database, distributed databases, cloud databases, and/or the like.

Server 706 may execute AI error detection system 102 described in FIG. 1. In some implementations, AI error detection system 102 may retrieve data, including AI generated content and source content from database 708 in real-time or at predefined time intervals. The error detection module 106 of AI error detection system 102 may identify errors in AI generated content and/or may transmit the AI generated content for display on AI interface 104 executing on user device 702. The AI interface 104 may display he AI generated content and underlying supporting source content for review by the user 704. Once AI interface 104 receives changes to AI generated content, the changes may be transmitted back to AI error detection system 102 on server 706 or stored in database 710. Similarly, error auditor 110 may retrieve the modified AI generated content from database 110 and generate audit report on server 106 and then transmit the audio report for display on AI interface 104.

Server 706 may include at least one network interface component 716 adapted to communicate with user device 702 and other devices, databases, etc., connected to network 712. In various embodiments, network interface component 716 may comprise a DSL (e.g., Digital Subscriber Line) modem, a PSTN (Public Switched Telephone Network) modem, an Ethernet device, a broadband device, a satellite device and/or various other types of wired and/or wireless network communication devices including microwave, radio frequency (RF), and infrared (IR) communication devices.

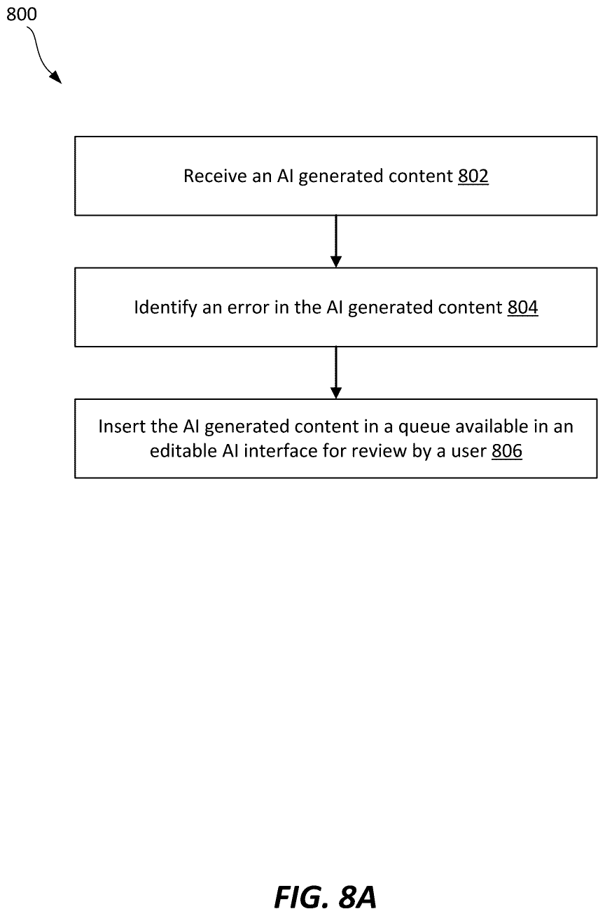

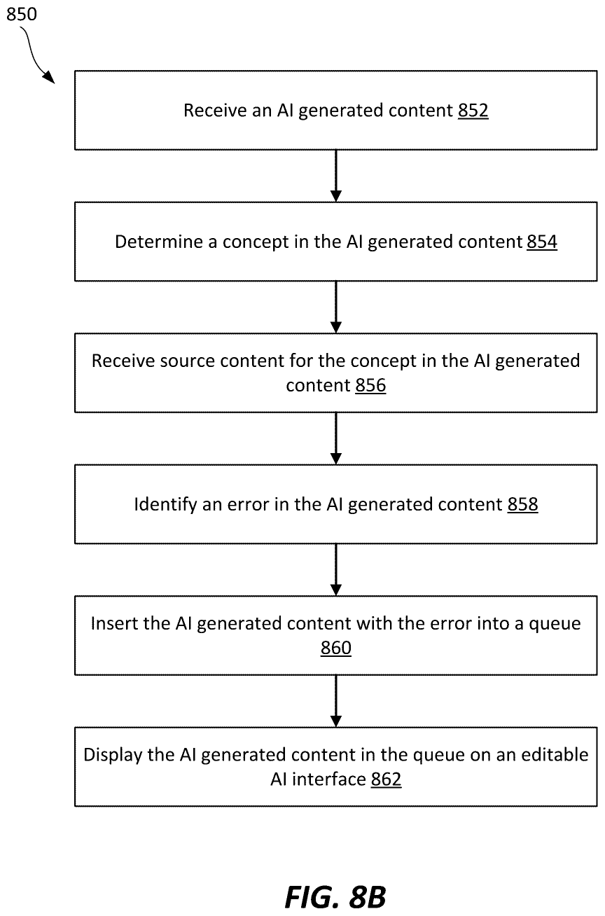

FIG. 8A is a flowchart of a method 800 for identifying an error in AI generated content according to some embodiments. One or more of the processes 802-806 of method 800 may be implemented, at least in part, in the form of executable code stored on non-transitory, tangible, machine-readable media that, when run by one or more processors, may cause the one or more processors to perform one or more of the processes 802-806. Method 800 may occur as AI generated content is generated from source content. AI error detection system 102 may then process the AI generated content to identify errors therein.

At operation 802, AI generated content is received. For example, server 706 may receive AI generated content from AI content generator 101. AI generated content may include patient summaries, discharge barriers, physician's notes, and other types of AI generated content that was generated using one or more neural networks, large language models, and the like. AI generated content may be received in real-time, i.e., as it is generated, at predefined time intervals or stored for later evaluation. In some instances, AI generated content may be an updated AI generated content generated in response to new source content becoming available. The method 800 may also be applied to the updated AI generated content. Furthermore, if no error is identified in the updated AI generated content, then the earlier version of the AI generated content where, for sake of argument, an error was identified may be removed from the queue designated for further review.

At operation 804, an error is identified in AI generated content. For example, AI error detection system 102 determines there is an error in the AI generated content. Identifying an error in AI generated content may include one or more steps. For example, AI hallucination module 112 may determine if a trigger word is present in the AI generated content. Once it is determined that a trigger word is present, error detection module 106 may parse the AI generated content into a plurality of concepts and/or treatments and query a database 708, 710 for relevant source content for each concept and/or treatment. Then error detection module 106 may use the relevant source content to the parsed concepts and/or treatments to determine/identify if there is an error in AI generated content.

At operation 806, AI generated content is inserted in a queue. The queue may be accessible to AI interface 104 that may upload the queue for display on a user interface of user device 702 and allow a user, such as a medical professional or clinician, to review the AI generated content. In other words, the server 706 may identify and/or provide the AI generated content for review to the user device 702, which may be associated with a user and/or user identifier. AI generated content may be displayed and edited via AI interface 104. The AI generated content, after modification, may be provided back to AI content generator 101 and used for training/fine-tuning one or more neural networks, large language models, and/or prompts.

FIG. 8B is a diagram of a method 850 for identifying an error in artificial intelligence generated content, according to some embodiments. One or more of the processes 852-862 of method 850 may be implemented, at least in part, in the form of executable code stored on non-transitory, tangible, machine-readable media that, when run by one or more processors, may cause the one or more processors to perform one or more of the processes 852-862. Method 850 may execute as AI generated content is generated from source content and is fed into, or accessed by AI error detection system 102. AI error detection system 102 may then process the AI generated content to identify errors therein.

At operation 852, AI generated content is received. For example, server 706 may receive AI generated content from AI content generator 101. AI generated content may include patient summaries, discharge barriers, physician's notes, and other types of AI generated content that was generated using one or more neural networks, large language models, and the like. AI generated content may be received in real-time, i.e., as it is generated, at predefined time intervals or stored for later evaluation. In some instances, AI generated content may be an updated AI generated content generated in response to new source content becoming available. The method 850 may also be applied to the updated AI generated content. Furthermore, if no error is identified in the updated AI generated content, then the earlier version of the AI generated content where, for sake of argument, an error was identified may be removed from the queue designated for further review.

At step 854, a concept in the AI generated content is determined. For example, error detection module 106 in error detection system 106 may receive the AI generated content and a prompt. The prompt may be saved in a configuration file stores in a database 708, 710 or locally at the server 706. The prompt may include a request to identify one or more concepts in the AI generated content. The error detection module receives the prompt and the AI generated content, e.g., a patient summary, and then parse the AI generated content into a collection of concepts as described in FIG. 1. In some embodiment, error detection module 106 comprises one or more neural networks, such as a large language model. In some embodiments, error detection module 106 or hallucination detection module 112 may search AI generated content for trigger words stored in a pre-defined list of trigger words. Trigger words may indicate the severity of a condition present in medical records which indicates a heightened need for review, as described above with respect to FIGS. 1 and 5.

At step 856, source content for the concept in the AI generated content is received. For example, after parsing the AI generated content, server 706 may query one or more databases 708, 710 for source content associated with each of the concepts, which are then received by the server 706 through the network 712. The source content may include structured and unstructured data that comprises electronic medical records, physician's notes, electronic health records, and the like that are associated with a patient.

At step 858, an error in the AI generated content is identified. For example, using the received source content, error detection module 106 may compare the concepts in the AI generated content with the received source content to determine factual accuracy. Tables I-III, above, provide non-limiting examples of the types of error that may be identified by error detection module 106 when concepts are compared to the AI generated content. In some instances, error detection module 106 may use a neural network model and/or LLM to receive a prompt that includes source content, AI generated content, and possible error types listed in Tables I-III as a prompt, and generate an output that identifies AI generated content that includes errors. If an error is detected, then error detection module 106 may flag the AI generated content for further review.

At step 860, the AI generated content with the error is inserted into a queue. For example, after flagging the AI generated content with an identified error may place the content into a queue with other flagged content. The queue may be stored locally at the server 706 or pushed to the user device 702 for particular users to review.

At step 862, the AI generated content in the queue is displayed on an editable AI interface. For example, once a user 704 has logged into their user device 702, the server receives a message notifying it that a reviewer is available to review flagged content. Server 706 may send one or more flagged AI generated content through the network 712 to the user device 702. On the user device 702, flagged AI generated content may be displayed to the user 704 through the AI interface 104 along with the source content. In some embodiment, AI interface 104 may be editable by a user 704. A user may provide instructions to modify the AI generated content, such as adding and/or removing text from AI generated content, through an editable AI interface 104. In some embodiment, the modified AI generated content is provided to another user. For example, a doctor or nurse in the normal course of evaluating a patient may receive a modified patient summary that was originally generated by AI content generator 101, flagged by the error detection module 106, and modified by a reviewer 704.

In some embodiments, modified AI generated content may be provided to the AI content generator to further train/finetune the neural networks therein. The training and structure of neural networks are described below in FIGS. 12-13.

In some embodiments, modified AI generated content may be provided recursively to the error detection system 102 to either confirm no more errors remain or to detect additional errors that must be corrected. One or more of the modules in AI error detection system 102 may be used in each recursive loop to search for additional errors. In some instances, prompts may be varied during the recursion to further reduce the risk of errors remaining in the AI generated content. Once the error detection system 102 determines that no errors remain in the AI generated content, then the flag may be removed from the content and/or the AI generated content may be removed from a review queue.

In some embodiments, new source content, e.g., medical records, may become available. Once the server 706 receives a notification that new source content is available, previously modified AI generated content may be reevaluated using the AI error detection system 102. In some instances, AI generated content currently in the queue may be reevaluated by the error detection system 102 considering the new source content. If the error detection module 106 or hallucination module 112 does not detect an error, then the AI generated content is removed from the queue considering the new source content.

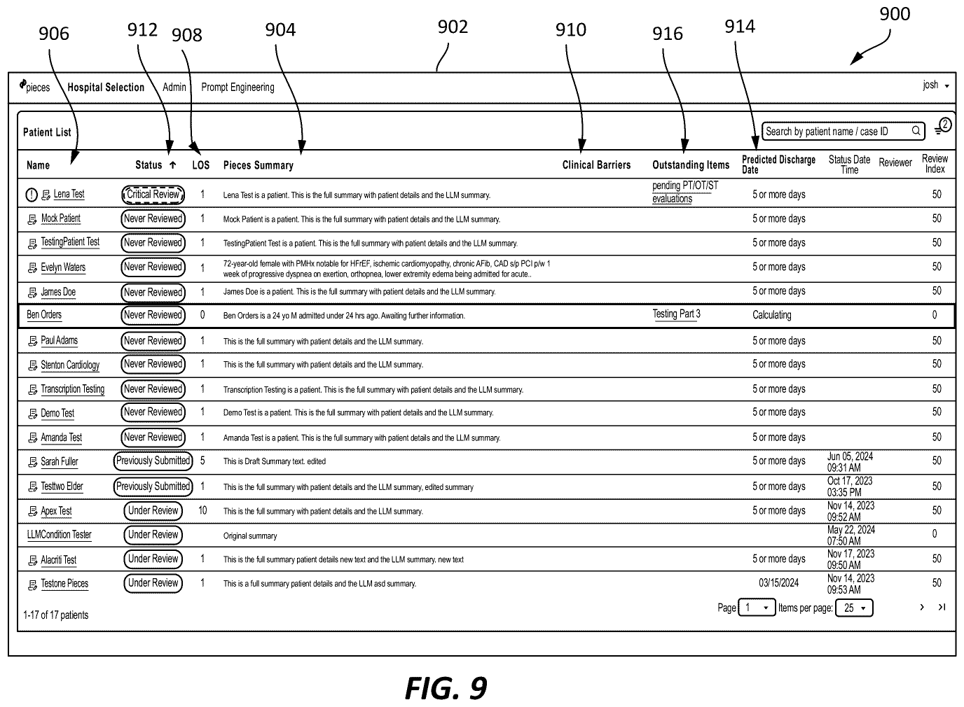

FIGS. 9-11B depict user interface capable of displaying AI generated content and/or corrected AI generated content generated by the AI error detection system 102. User interface may be configured to size and position material based on the screen size, device, or other settings of the device being operated by a user. The resizing and positioning is similar to that described with respect to FIGS. 2-4, above.

FIG. 9 is a diagram 900 of an artificial intelligence user interface, according to some embodiments. FIG. 9 may include an AI user interface 902 which may be AI interface 104. AI user interface 902 may receive and display AI generated content associated with multiple patients. AI user interface 902 may format the AI generated content in a list or another format. For example, AI user interface 902 may display a summary view of the patient data from multiple patients. The patient data may include a portion of AI generated content, such as a portion of the text summary in column 904 and patient names that corresponds to the AI generated content included in the name column 906. Additionally, AI user interface 902 may include a status column 912 that indicates the review status of the AI generated content. The review status may indicate whether the AI generated content was previously reviewed by user 108 (e.g., the reviewer) or error detection module 106, is under review, is saved for latter, or previously submitted by a reviewer.

AI user interface 902 may also include a clinical barrier column 910. The clinical barrier, also called a discharge barrier, column 910 displays the reasons or a portion of the reasons, if any, that prevent discharge of the patient as determined by the AI content generator 101. Additionally, AI user interface 902 may include a length-of-stay (LOS) column 908 that indicates how long a patient has been at a medical facility since their most recent admission, and projected discharge date column 914 that indicates the patient's projected discharge date.

Additionally, AI user interface 902 may include an outstanding items column 916 that indicates tests, evaluations, etc. that are pending completion or results for a patient. This data may be received over network 712 as the data becomes available.

The data displayed in the AI user interface 902 may be sorted or searched based on the information displayed in the various columns 904-916. For example, AI user interface 902 may receive input for sorting data based on the clinical barrier column 910 or projected discharge date column 914 to identify AI generated content that may be more time sensitive for review than other AI generated content.

The AI generated content may be uploaded to AI user interface 902 from a database or another memory storage. The AI generated content may be uploaded when AI user interface 902 is accessed or AI error detection system 102 is activated. Additionally, AI user interface 902 may be updated in real-time or at predefined time intervals when new AI generated content is saved to the database by, e.g., AI content generator 101.

Notably, the columns 904-916 displayed by AI user interface 902 are exemplary, as the columns of AI user interface 902 may be configured to reflect the project name, status, project duration, or summary of current status for use cases in other industries.

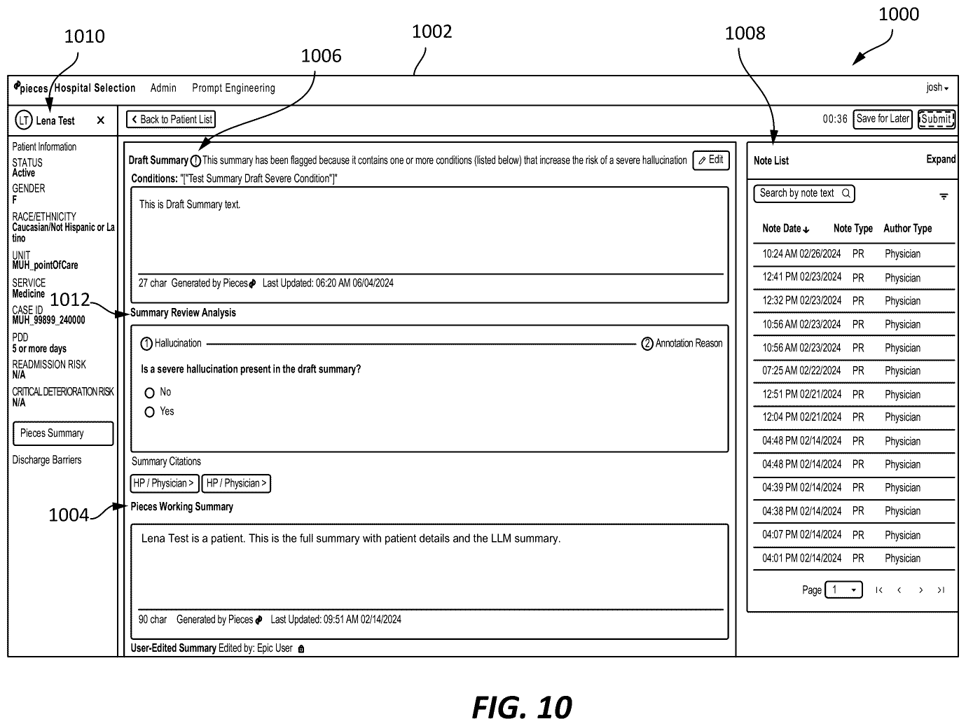

FIG. 10 is a diagram 1000 of an artificial intelligence user interface 1002, according to some embodiments. AI user interface 1002 may receive input from a user, such as a reviewer, and display AI generated content. AI user interface 1002 may be accessed from the AI user interface 902 by selecting data displayed in AI user interface 902, such as a portion of the text summary in column 904. AI user interface 1002 shown in FIG. 10 may display data associated with a patient, including AI generated content. An example of the AI generated content may be a summary of the patient's conditions. AI generated content may be displayed in a first text box or a window 1006. Additionally, AI user interface 1002 may include a second window or a text box 1004 where AI generated content 1006 may be modified via interface 1002, such as a modified AI generated summary of the patient conditions. The second window or text box 1004 may receive input via a stylus, keyboard, or another input device that may modify the AI generated content. Additionally, the second window or text box 1004 may receive the modified AI generated content that was automatically modified using error detection module 106. After modifications, AI user interface 1002 may submit the modified AI generated content to AI content generator 101 discussed above, for subsequent training or finetuning.

In some embodiments, AI user interface 1002 may include a note view 1008. Note view 1008 may display source content in a third text box or a window. In other use cases, not view may display financial or other company records generated by a department within a company. The notes may be source content, e.g., original notes as entered by the treating physician or nurse, images of the notes (not shown), or a subset of data in the notes that was tagged as relevant to determining AI generated content displayed in the text box 1004.

AI user interface 1002 may include an information panel 1010 in another text box or window. Information panel 1010 may display patient information, as depicted in FIG. 10, or other types of information for various use cases. For example, information panel 1010 may provide information about a company building project, including percent completion, start date, target completion date, and/or predicted probability of on-time completion. It should be appreciated that any combination of any subset of text boxes or windows described herein may be implemented.

In some embodiments, AI user interface 1002 may include a summary review analysis 1012. Summary review analysis 1012 may displays questions for the reviewer in another text box or window. The displayed questions may ask the user questions about the types of errors or hallucinations in the AI generated content 1006. Summary review analysis 1012 may update after each question is answered, showing a new question based on the response to the previous question. In some instances, an editable text box may be provided to the user to allow for explanations to entered in response to prompts. For example, a user may be asked to describe the type of hallucination that occurred, e.g., as described in the Tables I-III above.

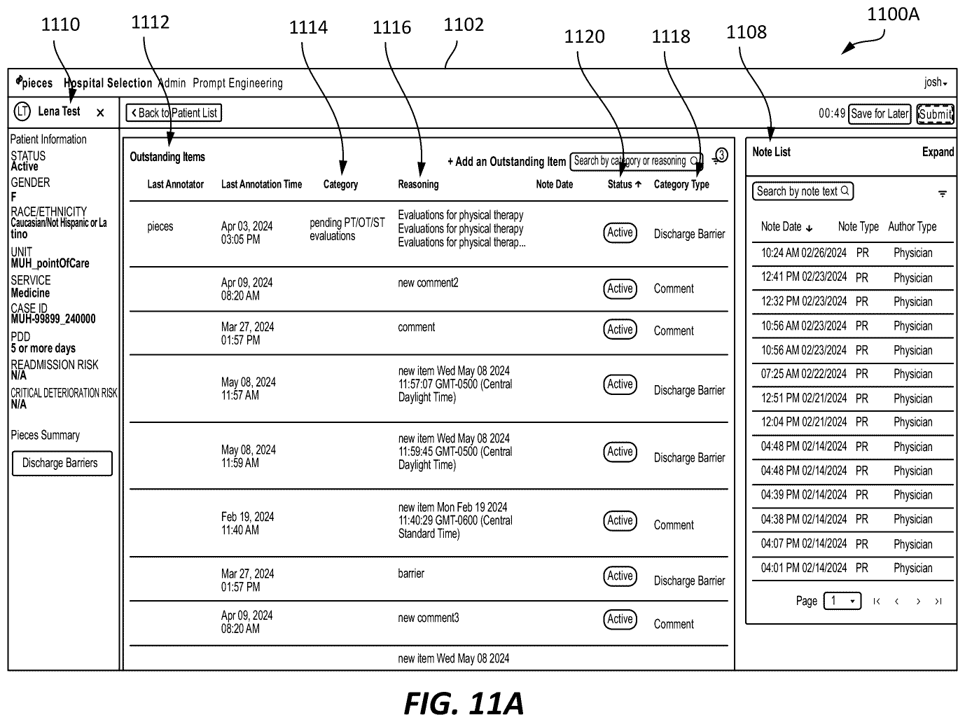

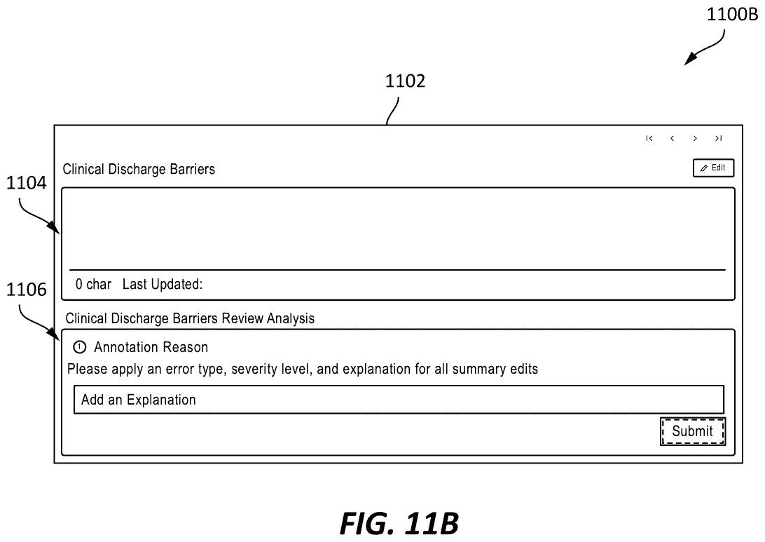

FIGS. 11A and 11B are diagrams 1100A and 1100B of an artificial intelligence user interface 1102, according to some embodiments. AI user interface 1102 may receive input from a user, such as a reviewer, and display AI generated content. AI user interface 1102 may be accessed from the AI user interface 902 by selecting data displayed in AI user interface 902, such as a portion of the text summary in column 904. AI user interface 1102 shown in FIGS. 11A-B may display data associated with a patient and discharge barriers associated with the patient, including AI generated content. AI generated content may be displayed in a first text box or a window 1104. Additionally, AI generated content in window 1104 may be modified, such as a modified AI generated discharge barrier for the patient. The window or text box 1104 may receive input via a stylus, keyboard, or another input device that may modify the AI generated content. Additionally, the window or text box 1104 may receive the modified AI generated content that was automatically modified using error detection module 106. After modifications, AI user interface 1102 may submit the modified AI generated content to AI content generator 101 discussed above, for subsequent training or finetuning.

In some embodiments, AI user interface 1102 may include a clinical discharge barriers review analysis window 1106. Clinical discharge barriers review analysis window 1106 may display questions for the reviewer in another text box or window. The displayed questions may ask the user questions about the types of errors/hallucinations in the AI generated content 1104. Clinical discharge barriers review analysis window 1106 may update after each question is answered, showing a new question based on the response to the previous question. In some instances, an editable text box may be provided to the user to allow for explanations to entered in response to prompts. For example, a user may be asked to describe the type of hallucination that occurred, e.g., as described in the Tables I-III above, the severity of error, and/or an explanation for any annotations made by the user.

In some embodiments, AI user interface 1102 may include a note view 1108. Note view 1108 may display source content in a third text box or a window. In other use cases, note view may display financial or other company records generated by a department within a company. The notes may be source content, e.g., original notes as entered by the treating physician or nurse, images of the notes (not shown), or a subset of data in the notes that was tagged as relevant to determining AI generated content displayed in the text box 1104.

AI user interface 1002 may include an information panel 1110 in another text box or window. Information panel 1110 may display patient information, as depicted in FIG. 11A, or other types of information for various use cases. For example, information panel 1110 may provide information about a company building project, including percent completion, start date, target completion date, and/or predicted probability of on-time completion. It should be appreciated that any combination of any subset of text boxes or windows described herein may be implemented.

AI user interface 1102 may format the AI generated content in a list or another format. For example, Additionally, AI user interface 1102 may include an outstanding items window 1112 that lists items that are unresolved for a patient. The review status may indicate whether may indicate whether an item has been resolved or not. The outstanding items may be generated by AI content generator 101 and prevent discharge of the patient. Additionally, AI user interface 1102 may include a category column 1114 that indicates a category for the outstanding item, e.g., evaluations are pending for physical therapy or some other treatment plan. AI user interface 1102 may include a reasoning column 1116 that further explains the reason for the discharge barrier. Reasoning column 1116 may be AI generated content produced by the AI content generator 101. AI user interface may include a category type column 1118 categorizing the note, e.g., as a “Discharge Barrier” note or a comment.

The data displayed in the AI user interface 1102 may be sorted or searched based on the information displayed in the various columns 1104-1120. For example, AI user interface 1102 may receive input for sorting data based on a status column 1120 or category type column 1118 to identify AI generated content that may be more time sensitive for review than other AI generated content.

The AI generated content may be uploaded to AI user interface 1102 from a database or another memory storage. The AI generated content may be uploaded when AI user interface 1102 is accessed or AI error detection system 102 is activated. Additionally, AI user interface 1102 may be updated in real-time or at predefined time intervals when new AI generated content is saved to the database by, e.g., AI content generator 101.

Notably, the columns 1112-1120 displayed by AI user interface 1102 are exemplary, as the columns of AI user interface 1102 may be configured to reflect the project name, status, project duration, or summary of current status for use cases in other industries. For example, a construction company may have several ongoing construction projects at varying stages of completion and faced with varying challenges (e.g., labor shortages, city ordinance restrictions, or extended/delayed contractual negotiations).

FIG. 12 is a simplified diagram illustrating the neural network structure that may be implemented in AI content generator 101 and/or AI error detection system 102, according to some embodiments. AI content generator 101 and/or AI error detection system 102 may include a perception neural network, a feed forward neural network, a multilayer perceptron network, a convolutional neural network, a radial basis functional neural network, a recurrent neural network, an LSTM (Long Short-Term Memory) network and the like. In some instances, AI content generator 101 and/or AI error detection system 102 may be implemented as a generative pre-trained transformer (GPT) model, large language model (LLM), or a bi-directional encoder representations from transformers (BERT) model.

AI content generator 101 and/or AI error detection system 102 may comprise neural network architecture. The example neural network architecture may comprise an input layer 1202, one or more hidden layers 1204 and an output layer 1206. The AI content generator 101 and/or AI error detection system 102 may be built as a collection of connected units or nodes, referred to as neurons 1208. Each layer 1202, 1204, or 1206 may comprise the same or different number of neurons or nodes 1208, with neurons between layers being interconnected according to a specific topology. Each neuron 1208 may be associated with an adjustable weight. The neurons 1208 may be aggregated into layers 1202, 1204, 1206 such that different layers may perform different transformations on the respective input to generate a transformed output, which is an input for the subsequent layer. Further, different layers in AI content generator 101 and/or AI error detection system 102 may be combined into their own neural network models, such that an output layer of one neural network model is an input into the next neural network model until a final output layer 1206 is reached.

Input layer 1202 receives input data, such as patient summaries or medical records from AI content generator 101 and/or AI error detection system 102. The number of nodes (neurons) in the input layer 1202 may be determined by the dimensionality of the input data (e.g., the length of a vector of a given example of the input). Each node 1208 in the input layer 1202 may represent a feature or attribute of the input. In some embodiments, input layer 1202 may be an embedding layer that may generate embeddings from input data. For example, words or tokens input data may be converted into vectors of fixed size called embedding vectors. The embedding vectors are mapped into a high-dimensional space. Additionally, positional encodings are added to the embedding vectors that may preserve the order of words in the input. Thus, each word and/or number in the input data may be transformed into embedding vectors, with the position each word and/or number maintained using the positional embeddings.

The hidden layers 1204 are intermediate layers located between the input and output layers 1202, 1206 of the AI content generator 101 and/or AI error detection system 102. Although three hidden layers 1204 are shown, there may be any number of hidden layers in the AI content generator 101 and/or AI error detection system 102. Hidden layers 1204 may extract and transform the input data through a series of weighted computations and activation functions associated with individual neurons.

For example, the AI content generator 101 and/or AI error detection system 102 may receive prompts and data (e.g., patient summaries, medical records, etc.) at input layer 1202 and generate prompt-responsive text or classifications in an output of output layer 1206. To perform the transformation, each neuron 1208 receives input signals (which may be input to AI content generator 101 and/or AI error detection system 102 or output of the preceding layer), performs a weighted sum of the inputs according to weights assigned to each connection and then applies an activation function associated with the respective neuron 1208 to the result. The output of the neuron is passed to the next layer of neurons or serves as the final output of the network. The activation function may be the same or different across different layers 1202, 1204, 1206, and may be different at neurons 1208 within each layer. Example activation functions include but are not limited to Sigmoid, hyperbolic tangent, Rectified Linear Unit (ReLU), Leaky ReLU, softmax, and/or the like. In this way, input data received at the input layer 1202 is transformed by hidden layers 1204 into different values indicative of data characteristics corresponding to a task that the AI content generator 101 and/or AI error detection system 102 has been trained to perform. Furthermore, the hidden layers may be organized and connected into larger network structures as described below.

Referring again to FIG. 12, the output layer 1206 is the final layer of the neural network structure. It produces the network's output or prediction based on the computations performed in the preceding layers (e.g., 1202, 1204). The number of nodes in the output layer depends on the nature of the task being addressed. For example, in a binary classification problem, the output layer may consist of a single node representing the probability of belonging to one class. In a multi-class classification problem, the output layer may have multiple nodes, each representing the probability of belonging to a specific class. In the embodiments discussed herein, an output of output layer 1206 may be a classification of a potential error in the AI generated content, such as errors discussed in Tables I-III above, an error warning or a revised patient summary.

Referring now to FIG. 13, which is a diagram 1300 of a large language model (LLM) for text generation and/or error detection, according to some embodiments. The LLM may receive an AI generated summary and/or prompt as input and generate an error, hallucination type, and/or an explanation as output.

The LLM model may include an input embedder 1310. Input embedder 1310 may be part of input layer 1202. Input embedder 1310 may receive input text, such as an AI generated patient summary and/or prompt 1305, tokenize the input text into tokens, and generate embedding vectors for the tokens that capture semantic and syntactic information from the input text. There may be one embedding vector for one token, where a token may represent a word in the input text. In some instances, positional encodings 1315_1 may be added to the embedding vectors to provide information of the positions of the tokens in the input text with respect to other tokens.

In some embodiments, one or more hidden layers 1204 may further be combined into layers and/or blocks. Example layers may be an encoder 1330, a decoder 1340, a linear layer 1360, and a softmax layer 1370. In a non-limiting embodiment, encoder 1330 may include a multi-head attention layer 1332_1, one or more normalization layers 1320, such as normalization layers 1320_1 and 1320_2, and a feed forward network 1334_1. Decoder 1340 may include a masked multi-head attention layer 1352, one or more normalization layers 1320, such as normalization layers 1320_3, 1320_4, and 1320_5, multi-head attention layer 1332_2, and feed-forward layer 1334_2. The encoder 1330 and decoder 1340 may comprise transformer blocks. Further, the outputs of one layer may be inputs into the subsequent layer as shown in FIG. 13.

Encoder 1330 may receive the embedding vectors from input embedder 1310 and pass the embedding vectors through multi-head attention layer 1332_1, normalization layers 1320_1 and 1320_2 and feed forward layer 1334 to generate hidden states that include the context and meaning of the input text. The multi-head self-attention layer 1332_1 may focus on different embedding vectors and identify the importance of different tokens in the input text. The feed forward layer 1334_1 may include two linear layers, with each layer including activation functions at its neurons. The neurons of each linear layer of feed forward layer 1334_1 may receive input from all neurons of the previous linear layer. The feed forward layer 1334_1 may capture interactions between tokens in input text. Normalization layers 1320_1 and 1320_2 may receive the output of the previous layer as input, e.g., the output multi-head self-attention layer 1332_1 and feed forward layer 1334_1 respectively and normalize the input. Normalizing the input may ensure that the output of a preceding layer has a consistent distribution. The output of encoder 1330 may be the output of normalization layer 1320_1. As illustrated in FIG. 13, multiple layers in encoder 1330 may receive the embedding vectors and the outputs of the preceding layer.

Decoder 1340 may receive an output of encoder 1330 and embedding vectors of output embedder 1350. Output embedder 1350 may receive input text, which may be a shifted output 1352 of the LLM. Shifted output 1352 may be error classification, hallucination type and/or an explanation 1380 shifted by a certain number of tokens. Output embedder 1350 may convert the shifted output 1352 into tokens, and generate the embedding vectors from the tokens. In some instances, positional encodings 1315_2 may be added to the embedding vectors to provide information on the positions of the tokens in the shifted output 1352 with respect to other tokens.

Masked multi-head attention layer 1352 and normalization layer 1320_3 of decoder 1340 may receive the embedding vectors of the shifted output 1352. Masked multi-head attention layer 1352 may be a variant of multi-head attention layers 1332 where the prediction of output tokens depends on previous tokens because the embedding vectors that correspond to future tokens are masked. The output of the masked multi-head attention layer 1352 may also be fed into normalization layer 1320_3. Normalization layer 1320_3 may normalize its input and ensure that the embedding vectors of the shifted output 1352 and output of the masked multi-head attention layer 1352 have a consistent distribution.

The multi-head attention layer 1332_2 may receive the output of encoder 1330 and output of normalization layer 1320_3 and generate an output that focuses on an importance of different tokens in the input text (e.g., the patent summary and/or prompt 1305 and shifted output 1352). The output of the multi-head attention layer 1332_2 and normalization layer 1320_3 may be fed into the normalization layer 1320_4. Normalization layer 1320_4 may normalize its input, e.g., the output of the multi-head attention layer 1332_2 and the output of normalization layer 1320_3 to make sure the input has a consistent distribution.

Feed forward layer 1334_2 may capture interactions between tokens in input text and shifted output 1352 by processing the output of the normalization layer 1320_4 as input. Like feed forward layer 1334_1, feed forward layer 1334_2 may include two linear layers, with each layer including activation functions at their neurons. The neurons of each linear layer of feed forward layer 1334_2 may receive input from all neurons of the previous linear layer.

Normalization layer 1320_5 may receive the output of feed forward layer 1334_2 and output of normalization layer 1320_4 as input and normalize the output, which may be the output of decoder 1340.

Linear layer 1360 may receive the output of decoder 1340. Linear layer 1360 and softmax layer 1370 may be used to generate a probability distribution of a next token in the large language model output, which may be an error classification, hallucination type or an explanation 1380. Linear layer 1360 may be a fully connected layer where all neurons of the linear layer receive inputs from a preceding layer, e.g., normalization layer 1320_5, and apply linear transformation to the inputs by applying corresponding weights of the neurons and adding bias. The output of linear layer 1360 may be an input to softmax layer 1370 that generates an error classification, hallucination type or an explanation 1380. Softmax layer may be an output layer 1206 in FIG. 12 which generates probability distributions for next tokens, e.g., a words, to be included in error classification, hallucination type or an explanation 1380. As discussed above, error classification, hallucination type or an explanation 1380 may be shifted to be fed as input into decoder 1340.

Going back to FIG. 12, AI content generator 101 and/or AI error detection system 102 may also be implemented by hardware, software, and/or a combination thereof. For example, AI content generator 101 and/or AI error detection system 102 may comprise a specific neural network structure implemented and run on various hardware platforms, such as but not limited to CPUs (central processing units), GPUs (graphics processing units), FPGAs (field-programmable gate arrays), Application-Specific Integrated Circuits (ASICs), dedicated AI accelerators like TPUs (tensor processing units), and specialized hardware accelerators designed specifically for the neural network computations described herein, and/or the like. Example specific hardware for neural network structures may include, but not limited to Google Edge TPU, Deep Learning Accelerator (DLA), NVIDIA AI-focused GPUs, and/or the like. The hardware may be used to implement the neural network structure is specifically configured based on factors such as the complexity of the neural network, the scale of the tasks (e.g., training time, input data scale, size of training dataset, etc.), and the desired performance.

AI content generator 101 and/or AI error detection system 102 may be trained by iteratively updating the underlying weights of the neurons 1208, etc., bias parameters and/or coefficients in the activation functions associated with neurons 1208. The weights may be updated based on a loss function, such as a mean squared estimation error (MSEE), cross-entropy loss, log-loss, and the like. For example, during training, the training data such historical signals are fed into AI content generator 101 and/or AI error detection system 102 over thousands of iterations. The training data flows through the network's layers 1202, 1204, 1206, with each layer performing computations based on its weights, biases, and activation functions until the output layer 1206 produces the output.

The training data may be labeled with an expected output (e.g., a “ground-truth” such as a corresponding ground truth label). For example, training data that includes content may be labelled to have various comprehension type errors, fluency type errors, and total summarization type errors discussed in tables I-III. The output generated by the output layer 1206 is compared to the expected output from the training data to compute a loss function that measures the discrepancy between the predicted output and the expected output. In some embodiments, the negative gradient of the loss function may be computed with respect to the weights of each layer individually. This negative gradient is computed one layer at a time, iteratively backward from the last layer 1206 to the input layer 1202 of the AI content generator 101 and/or AI error detection system 102. These gradients quantify the sensitivity of the network's output to changes in the parameters. The chain rule may be applied to efficiently calculate these gradients by propagating the gradients backward (in a back propagation network) from the output layer 1206 to the input layer 1202.

Parameters of the neural network are updated backwardly from the last layer to the input layer (backpropagating) based on the computed negative gradient using an optimization algorithm to minimize the loss. The backpropagation from the last layer 1206 to the input layer 1202 may be conducted for a number of training samples in a number of iterative training epochs. In this way, parameters of the AI content generator 101 and/or AI error detection system 102 may be gradually updated in a direction to result in a lesser or minimized loss, indicating the neural network has been trained to generate a predicted output value closer to the target output value with improved prediction accuracy. Training may continue until a stopping criterion is met, such as reaching a maximum number of epochs or achieving satisfactory performance on the validation data. In a multiple neural network embodiment, the neural network models may be trained separately and then combined together and trained as a single AI content generator 101 and/or AI error detection system 102.

Neural network parameters may be trained over multiple stages. For example, initial training (e.g., pre-training) may be performed on one set of training data, and then an additional training stage (e.g., fine-tuning) may be performed using a different set of training data, such as machine-readable code in one or more programming languages. In some embodiments, all, or a portion of parameters of one or more neural-network models being used together may be frozen, such that the “frozen” parameters are not updated during that training phase. This may allow, for example, a smaller subset of the parameters to be trained without the computing cost of updating all the parameters.

Therefore, the training process transforms the neural network into an “updated” trained neural network with updated parameters such as weights, activation functions, and biases. The trained neural network thus improves neural network technology in text summarization and error detection in AI generated content. Other AI generated content may serve as input as described herein, e.g., physician's notes, discharge barriers, etc. In some embodiments, individual modules, e.g., the AI hallucination detection module 112, may implement the neural network structure as described herein.

Once training is complete, the trained AI content generator 101 and/or AI error detection system 102 may enter an inference stage where AI content generator 101 and/or AI error detection system 102 may be used to make predictions on new, unseen data, such as generating patient summaries or detecting errors based on prompts that include patient medical records, patient summaries, etc.

Therefore, the training process transforms the neural network into an “updated” trained neural network with updated parameters such as weights, activation functions, and biases.

Although an exemplary embodiment of at least one of a system, method, and non-transitory computer readable medium has been illustrated in the accompanying drawings and described in the foregoing detailed description, it will be understood that the application is not limited to the embodiments disclosed, but rather is capable of numerous rearrangements, modifications, and substitutions as set forth and defined by the following claims. For example, the capabilities of the system of the various figures can be performed by one or more of the modules or components described herein, or in a distributed architecture, and may include a transmitter, receiver, or pair of both. For example, all or part of the functionality performed by the individual modules may be performed by one or more of these modules. Further, the functionality described herein may be performed at various times and in relation to various events, internal or external to the modules or components. Also, the information sent between various modules can be sent between the modules via at least one of: a data network, the Internet, a voice network, an Internet Protocol network, a wireless device, a wired device and/or via plurality of protocols. Also, the messages sent or received by any of the modules may be sent or received directly and/or via one or more of the other modules.

One skilled in the art will appreciate that a “system” could be embodied as a personal computer, a server, a console, a personal digital assistant (PDA), a cell phone, a tablet computing device, a smartphone or any other suitable computing device, or combination of devices. Presenting the above-described functions as being performed by a “system” is not intended to limit the scope of the application in any way but is intended to provide one example of many embodiments. Indeed, methods, systems and apparatuses disclosed herein may be implemented in localized and distributed forms consistent with computing technology.

It should be noted that some of the system features described in this specification have been presented as modules, in order to more particularly emphasize their implementation independence. For example, a module may be implemented as a hardware circuit comprising custom very large-scale integration (VLSI) circuits or gate arrays, off-the-shelf semiconductors such as logic chips, transistors, or other discrete components. A module may also be implemented in programmable hardware devices such as field programmable gate arrays, programmable array logic, programmable logic devices, graphics processing units, or the like.

A module may comprise any hardware or software-based framework that includes any artificial intelligence network or system, neural network or system and/or any training or learning models implemented thereon or therewith.

A module may also be at least partially implemented in software for execution by various types of processors. An identified unit of executable code may, for instance, comprise one or more physical or logical blocks of computer instructions that may, for instance, be organized as an object, procedure, or function. Nevertheless, the executables of an identified module need not be physically located together but may comprise disparate instructions stored in different locations which, when joined logically together, comprise the module and achieve the stated purpose for the module. Further, modules may be stored on a computer-readable medium, which may be, for instance, a hard disk drive, flash device, random access memory (RAM), tape, or any other such medium used to store data.

Indeed, a module of executable code could be a single instruction, or many instructions, and may even be distributed over several different code segments, among different programs, and across several memory devices. Similarly, operational data may be identified and illustrated herein within modules and may be embodied in any suitable form and organized within any suitable type of data structure. The operational data may be collected as a single data set or may be distributed over different locations including over different storage devices, and may exist, at least partially, merely as electronic signals on a system or network.

The features, structures, or characteristics of the application described throughout this specification may be combined in any suitable manner in one or more embodiments. For example, the usage of the phrases “example embodiments”, “some embodiments”, or other similar language throughout this specification refers to the fact that a particular feature, structure, or characteristic described in connection with the embodiment may be included in at least one embodiment of the application. Thus, appearances of the phrases “example embodiments”, “in some embodiments”, “in other embodiments”, or other similar language, throughout this specification do not necessarily all refer to the same group of embodiments, and the described features, structures, or characteristics may be combined in any suitable manner in one or more embodiments.

It will be readily understood that the components of the application, as generally described and illustrated in the figures herein, may be arranged and designed in a wide variety of different configurations. Thus, the detailed description of the embodiments is not intended to limit the scope of the application as claimed but is merely representative of selected embodiments of the application.

One having ordinary skill in the art will readily understand that the above may be practiced with steps in a different order, and/or with hardware elements in configurations that are different than those which are disclosed. Therefore, although the application has been described based upon these preferred embodiments, it would be apparent to those of skill in the art that certain modifications, variations, and alternative constructions would be apparent.

While preferred embodiments of the present application have been described, it is to be understood that the embodiments described are illustrative only and the scope of the application is to be defined solely by the appended claims when considered with a full range of equivalents and modifications (e.g., protocols, hardware devices, software platforms etc.) thereto.

While example embodiments have been particularly shown and described, it will be understood by those skilled in the art that various changes in form and details may be made therein without departing from the scope of the embodiments encompassed by the appended claims.

What is claimed is:

1. A system comprising:
a memory configured to store instructions for an artificial intelligence (AI) system; and
a processor coupled to the memory and configured to read the instructions from the memory to cause the system to perform operations, the operations comprising:
receiving an AI generated content at a neural network;
determining, using the neural network and at least one prompt, a concept in the AI generated content;
receiving source content for the concept in the AI generated content;
identifying, using the neural network, the source content, the concept, and a list of error types, an error in the AI generated content; and
inserting the AI generated content with the error into a queue, wherein the AI generated content in the queue is displayed on an editable AI interface.

2. The system of claim 1, wherein the operations further comprise:
displaying the AI generated content on the editable AI interface;
receiving, via the editable AI interface, instructions to modify the AI generated content based on the error; and
providing the modified AI generated content for display on another user device.

3. The system of claim 2, wherein the operations further comprise:
providing the modified AI generated content to at least one neural network in a plurality of neural networks for retraining the at least one neural network using the modified AI generated content.

4. The system of claim 1, wherein the operations for identifying the error in the AI generated content further comprise:
querying a database for the source content that includes the concept; and
comparing a concept in the source content with the concept in the AI generated content to identify the error.

5. The system of claim 1, wherein the neural network is a language model.

6. The system of claim 1, wherein the operations further comprise:
identifying one or more trigger words within the AI generated content, wherein the one or more trigger words are in a pre-defined list.

7. The system of claim 1, wherein operations further comprise:
receiving an updated AI generated content that corresponds to the AI generated content with the error;
identifying that there is no error in the updated AI generated content; and
removing the AI generated content from the queue, wherein the updated AI generated content is generated in response to new source content available in a database.

8. A method comprising:
determining, using a neural network executing on a processor and at least one prompt, a concept in an AI generated content;
querying a database for source content from which the AI generated content was generated, wherein the source content is associated with the concept;
identifying, using the neural network, the source content, and the concept, an error in the AI generated content; and
inserting the AI generated content with the error into a queue, wherein the AI generated content in the queue is displayed on an editable AI interface.

9. The method of claim 8, further comprising:
displaying the AI generated content on an editable AI interface; and
modifying, using the editable AI interface, the AI generated content based on the error.

10. The method of claim 9, further comprising:
providing the modified AI generated content to the neural network for retraining the neural network using the modified AI generated content.

11. The method of claim 8, further comprising:
providing, to the neural network, a list of errors in a configuration file, wherein the list of errors includes the error.

12. The method of claim 8, further comprising:
identifying one or more trigger words within the AI generated content, wherein the one or more trigger words are in a pre-defined list; and
wherein the determining is based on identifying the one or more trigger words within the AI generated content.

13. The method of claim 8, further comprising:
receiving an updated AI generated content;
identifying that there is no error in the updated AI generated content; and
removing the AI generated content from the queue, wherein the updated AI generated content is generated in response to the database being updated with a new source content.

14. The method of claim 8, wherein the AI generated content comprises at least one of:
a patient summary;
a discharge barrier; or
a physician note.

15. A non-transitory computer-readable medium having instructions thereon, that when executed by a processor, cause the processor to perform operations comprising:
receiving an AI generated summary;
determining, using a neural network and at least one prompt, a concept in the AI generated summary;
querying a database for source content for the concept in the AI generated summary;
identifying, using the neural network, the source content, and the AI generated summary, an error in the AI generated summary; and
inserting the AI generated summary in a queue available in an editable AI interface.

16. The non-transitory computer-readable medium of claim 15, wherein the operations further comprising:
displaying the AI generated summary on the editable AI interface; and
receiving instructions to modify, using an editable AI interface, the AI generated summary based on the error.

17. The non-transitory computer-readable medium of claim 16, wherein the operations further comprising:
providing the modified AI generated summary to the neural network for retraining the neural network using the modified AI generated summary.

18. The non-transitory computer-readable medium of claim 17, wherein the AI generated summary is parsed by a language model.

19. The non-transitory computer-readable medium of claim 15, the operations further comprising:
identifying one or more trigger words within the AI generated summary, wherein the one or more trigger words are stored in a configuration file; and
identifying the error in the AI generated summary when the AI generated summary includes the one or more trigger words.

20. The non-transitory computer-readable medium of claim 15, the operations further comprising:
receiving an updated AI generated summary;
identifying that there is no error in the updated AI generated summary; and
removing the AI generated summary from the queue, wherein the updated AI generated summary is generated in response to new source content available in the database.
